(19) United States
(12) Patent Application Publication
(10) Pub. No.: US 2023/0184779 A1
(43) Pub. Date: Jun. 15, 2023

(54) METHODS OF DETECTING CLOSTRIDIUM DIFFICILE INFECTIONS

(71) Applicant: Washington University, St. Louis, MO (US)

(72) Inventors: Jeffrey P. Henderson, St. Louis, MO (US); John Isaac Robinson, St. Louis, MO (US); Jan Maureen Crowley, St. Louis, MO (US)

(21) Appl. No.: 17/614,140

(22) PCT Filed: May 29, 2020
(86) PCT No.: PCT/US2020/035352

§ 371 Date: Nov. 24, 2021

Related U.S. Application Data
(60) Provisional application No. 62/854,190, filed on May 29, 2019.

(51) Int. Cl. G01N 33/68

(52) U.S. Cl. CPC G01N 33/6812; G01N 33/6893; G01N 2800/26

(57) ABSTRACT

The present disclosure provides methods related to accurate detection and treatment of medical conditions related to Clostridioides difficile infection (CDI). In specific cases, the disclosure concerns accurate assessment of a symptom associated with CDI related to the presence or risk that may or may not be a pathogenic infection. Particular embodiments encompass detection of one or more specific analytes that provide information for accurate diagnosis and treatment of CDI.

### CROSS REFERENCE TO RELATED APPLICATION

This application claims the benefit of International Patent Application PCT/US2020/035352, filed May 29, 2020, which claims the benefit of U.S. Provisional Application 62/854,190, filed May 29, 2019, the disclosure of which is hereby incorporated by reference in its entirety.

### GOVERNMENT SUPPORT

This invention was made with government support under 200-2016-91965 awarded by the Centers for Disease Control and Prevention. The government has certain rights in the invention.

### FIELD OF THE TECHNOLOGY

This disclosure generally relates to the field include bacteriology, cell biology, physiology, molecular biology, microbial detection, and medicine.

### BACKGROUND

Clostridioides difficile infection (CDI) is listed by the CDC as an urgent threat to public health. Early CDI diagnosis is crucial for optimal clinical management and improved prognosis. Due to the rapid turn-around and cost effectiveness, many hospitals utilize nucleic acid amplification tests to diagnose CDI. However, such sensitive molecular testing is widely recognized to misdiagnose up to 30% of CDI cases. A major reason for this misdiagnosis is that a positive stool test cannot differentiate Clostridioides difficile (formerly known as Clostridium difficile) colonization from symptomatic disease. Underscoring the importance of this assay deficiency, other factors including younger age and non-responsiveness to CDI therapy positively correlate with higher rates of alternative diagnoses, e.g., functional gastrointestinal disorders (FGIDs), inflammatory bowel disease (IBD), non-CDI infectious colitis.

Thus, a need in the art exists for a robust CDI detection assay useful to guide treatment decisions.

### SUMMARY

The present disclosure is based on the finding of specific analyte signatures being useful for a robust CDI detection assay useful to guide treatment decisions. In some aspects, the present disclosure provides a method of identifying a subject having a Clostridioides difficile infection (CDI), by (i) measuring levels of one or more analytes selected from the group consisting of a short chain fatty acid, an amino acid, a bile acid, a carbohydrates, an aromatic alcohol and a lipid in a biological sample obtained from the subject; (ii) determining an analyte signature based on the expression levels of the analytes in step (i); and (iii) identifying CDI occurrence of the subject based on the analyte signature determined in step (ii). In some embodiments, the biological sample is a fecal sample. In some embodiments, the analyte levels are measured by mass spectrometry.

In some embodiments, the analytes are one or more of 4-methylpentanoic acid (4-MPA), 2-hydroxy-4-methylpentanoci acid, isoleucine, leucine, allo-isoleucine, cholenoic acid, ribitol, eicosatrienoic acid, tyrosol, glyceryl glycoside, 4-MPA/leucine ratio and fructose.

In some embodiments, the subject is identified as having or at risk for CDI and the method further comprises subjecting the subject to a treatment for CDI. In some embodiments, the subject has undergone a prior treatment for a bacterial infection. In some embodiments, increased analyte levels of 4-methylpentanoic acid (4-MPA), 2-hydroxy-4-methylpentanoci acid, or allo-isoleucine in step (i) relative to a reference value indicates the subject has CDI. In other embodiments, decreased analyte levels of isoleucine, leucine, cholenoic acid, ribitol, eicosatrienoic acid, tyrosol, glyceryl glycoside, or fructose relative to a reference value of a subject having transient synovitis indicates the subject has CDI. In still other embodiments, increased analyte levels of 4-methylpentanoic acid (4-MPA), 2-hydroxy-4-methylpentanoci acid, or allo-isoleucine and decreased analyte levels of isoleucine, leucine, cholenoic acid, ribitol, eicosatrienoic acid, tyrosol, glyceryl glycoside, or fructose in step (i) relative to a reference value indicates the subject has CDI. In still another embodiment, similar analyte levels of 4-methylpentanoic acid (4-MPA), 2-hydroxy-4-methylpentanoci acid, or allo-isoleucine in step (i) relative to a reference value indicates the subject does not have CDI or is colonized with CDI. In still yet another embodiment, similar analyte levels of isoleucine, leucine, cholenoic acid, ribitol, eicosatrienoic acid, tyrosol, glyceryl glycoside, or fructose relative to a reference value of a subject does not have CDI or is colonized with CDI.

In another aspect, the present disclosure provides a method of treating a subject having a Clostridioides difficile infection (CDI), by (i) measuring levels of one or more analytes selected from the group consisting of a short chain fatty acid, an amino acid, a bile acid, a carbohydrates, an aromatic alcohol and a lipid in a biological sample obtained from the subject; (ii) determining an analyte signature based on the expression levels of the analytes in step (i); (iii) assessing CDI occurrence or severity of the subject based on the analyte signature determined in step (ii); and (iv) treating the subject with an anti-CDI therapeutic when the analyte signature in step (i) relative to a reference value indicates the subject has CDI. In some embodiments, the biological sample is a fecal sample. In some embodiments, the analyte levels are measured by mass spectrometry.

In some embodiments, the subject has undergone a prior treatment for a bacterial infection.

In some embodiments, the analytes are one or more of 4-methylpentanoic acid (4-MPA), 2-hydroxy-4-methylpentanoci acid, isoleucine, leucine, allo-isoleucine, cholenoic acid, ribitol, eicosatrienoic acid, tyrosol, glyceryl glycoside, 4-MPA/leucine ratio and fructose. In some embodiments, increased analyte levels of 4-methylpentanoic acid (4-MPA), 2-hydroxy-4-methylpentanoci acid, or allo-isoleucine in step (i) relative to a reference value indicates the subject has CDI. In other embodiments, decreased analyte levels of isoleucine, leucine, cholenoic acid, ribitol, eicosatrienoic acid, tyrosol, glyceryl glycoside, or fructose relative to a reference value of a subject having transient synovitis indicates the subject has CDI. In still other embodiments, increased analyte levels of 4-methylpentanoic acid (4-MPA), 2-hydroxy-4-methylpentanoci acid, or allo-isoleucine and decreased analyte levels of isoleucine, leucine, cholenoic acid, ribitol, eicosatrienoic acid, tyrosol, glyceryl glycoside, or fructose in step (i) relative to a reference value indicates the subject has CDI.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A-1D show the metabolomic characteristics of the patient cohort. FIG. 1A is a histogram showing the distribution of feature richness (number of features present per sample) across all patient specimens. FIG. 1B is a histogram showing the number of samples within which each unique feature is present. Fecal metabolomes were highly individualistic: among the more than 2000 features detected, most were infrequent. While the resulting data are very sparse overall, the distribution has a relatively heavy tail with a few features present in many samples. FIG. 1C shows the principal component analysis (PCA) score plot across the first 2 components created using log-transformed feature intensities across all metabolomic features. FIG. 1D shows the PCA does not appear to reveal dominant modes of variation, with no single component explaining more than 9% of the variance and a long tail of modes each explaining approximately 1% each.

FIG. 2A-2D show supervised metabolomic analyses comparing Cx⁺/EIA⁺ with Cx⁻/EIA⁻ samples. FIG. 2A shows the observed separation of Cx⁺/EIA⁺ and Cx⁻/EIA⁻ samples under sparse partial least squares-discriminatory analysis (sPLS-DA). The data ellipses are drawn around each group of samples (at the 95% level). FIG. 2B shows the penalized logistic regression under repeated 5-fold cross-validation shows how the number of features used relates to the obtained accuracy, yielding high accuracy with a relatively small number of features. The maximum percent predicted is indicated by a star. FIG. 2C shows using the penalty parameter associated with the maximum percent predicted, penalized logistic regression demonstrates good separation in the distribution of log-odds to be classified Cx⁺/EIA⁺ versus Cx⁻/EIA⁻. In the log-odds distribution shown here, only the test folds of Cx⁺/EIA⁺ and Cx⁻/EIA⁻ for each randomized cross-validated run are shown (that is, the corresponding distribution of the training set is not shown). For comparison, the corresponding log-odds of the Cx⁺/EIA⁻ samples are also shown. FIG. 2D shows the logistic regression (without penalty) to classify Cx+/EIA+ versus Cx−/EIA− was performed using only the 6 features most frequently used in the penalized logistic regressions. Fitting to all samples gives 96.7% ROC AUC. The 95% CI of 85.6%-100% AUC was obtained under repeated randomized 5-fold cross-validation using the same 6 features.

FIG. 3A-3C show the amino acid metabolism in C. difficile. FIG. 3A shows Stickland metabolism consists of anaerobic amino acid fermentation through coupled oxidation and reduction pathways. In the reductive pathway, amino acids are first deaminated to form 2-hydroxy acids and then reduced to carboxylic acids. In the oxidative pathway, amino acids are deaminated and oxidized with loss of CO₂ to yield a distinct set of carboxylic acids. Depicted here are established Stickland substrates and products identified within patient fecal metabolomes. Stickland substrates include the nonproteinogenic amino acid ornithine. ND, not determined. FIG. 3B shows a heatmap of Stickland precursor and product abundances corresponding to patient fecal metabolomes from the 3 diagnostic groups. Metabolites were organized using unsupervised hierarchical clustering. Metabolites differing significantly (Mann-Whitney U test; *P<0.05, ***P<0.001) between Cx⁻/EIA⁻ and Cx⁺/EIA⁺ groups are labeled, along with the direction of the difference relative to the Cx⁻/EIA⁻ control group. Stickland products are labeled according to the color scheme in A. FIG. 3C shows adjusted and unadjusted (crude) CDI odds ratios and confidence intervals (95%) for Stickland precursors and products. Odds ratios were estimate by fitting logistic regression models to each of 2000 bootstrap samples stratified on Cx/EIA status (Cx⁻/EIA⁻ vs. Cx⁺/EIA⁺). Logistic models containing a single metabolite were fit to obtain crude odds ratios (red). A single logistic model including all metabolites was fit to obtain the adjusted odds ratios (green). Bars represent 95% bootstrap percentile confidence intervals and black dots represent median odds ratios across all bootstrap samples. Stickland products are labeled according to the color scheme in A.

FIG. 4A-4B are graphs showing the 4-MPA/leucine ratio elevated in CDI. FIG. 4A shows dot plots of 4-MPA/leucine product/precursor ratios measured by targeted (SIM) reanalysis of fecal specimens (n=32 for each group). Patient groups were compared using the Kruskal-Wallis test (P=1.3×10⁻⁸). To further characterize pair-wise differences between groups, Bonferroni-corrected Mann-Whitney U test P values are indicated (3 comparisons; NS: P≥0.05, ***P<0.001). Ratio thresholds giving perfect specificity (0.0825, black star) or sensitivity (0.00132, white star) for CDI⁺/EIA⁺ are marked as gray dashed lines. FIG. 4B shows receiver-operator characteristic (ROC) plot distinguishing Cx⁺/EIA⁺ patients from Cx⁻/EIA⁻ patients. The gray region represents the bootstrapped 95% confidence interval for the true-positive rate at each false-positive rate. Thresholds with perfect specificity or sensitivity are marked by stars, as in A.

FIG. 5A-5C show isoleucine isomer correlated with C. difficile. FIG. 5A shows chemical structures of isoleucine and its diastereomer, allo-isoleucine. FIG. 5B shows a dot plot of allo-isoleucine/isoleucine ratios as measured by SIM (n=32 for each group). Patient groups were compared using the Kruskal-Wallis test (P=6.5×10⁻⁵). To further characterize pair-wise differences between groups, Bonferroni-corrected Mann-Whitney U test P values are indicated (3 comparisons; NS: P≥0.05, ***P<0.001). FIG. 5C shows an ROC plot showing ability to distinguish Cx⁺/EIA⁺ patients from Cx⁻/EIA⁻ patients. The gray region represents the bootstrapped 95% confidence interval for the true-positive rate at each false-positive rate.

FIG. 6A-6B show bile acid transformations in the clinical cohort. FIG. 6A shows a force-directed network layout illustrates associations between bile acids in the study cohort. Each node represents a bile acid and each connecting line (edge) represents an association between 2 bile acids as 1 of the 5 highest correlations for at least 1 of the corresponding nodes. Edge lengths are determined by the level of correlation between connected bile acids. Nodes are colored by community assignment.

FIG. 6B shows a scheme showing metabolic transformations producing bile acids in the network analysis. The central structure highlighted in gray represents a tri-hydroxylated primary bile acid (e.g., cholic acid). Taurine or glycine conjugation forms peptide bonds to the carboxylic acid group (right). Alcohol groups are removed from the bile acid nucleus (dehydroxylation, bottom right) or oxidized to a ketone (top left). Bile acid sulfation involves substitution of an alcohol group with a sulfate (R=SO₄⁻) group (bottom left). Desulfation of bile acid sulfates yields unsaturated bile acids (left).

FIG. 7A-7D shoe the bile acid distribution in patients with CDI resembles that of a characteristic subgroup of uninfected, hospitalized patients. FIG. 7A shows a PCA plot of uninfected patients' bile acid profiles (green, n=62). Onto this space, we projected the bile acid metabolome of patients with CDI (red, n=62). Data ellipses are drawn around each group of samples (95% level). Clustering of CDI specimens at high PC1 values is consistent with a favored bile acid distribution among patients with CDI. FIG. 7B shows a dot plot of PC1 scores for each patient sample (n=62 in each group). Gray dashed line represents optimal PC1 threshold for distinguishing Cx⁻/EIA⁻ from Cx⁺/EIA⁺ samples. This threshold was chosen by maximizing the sum of percent sensitivity and specificity. FIG. 7C shows a ROC plot evaluating the ability of PC1 to distinguish CDI patients from controls. The gray region represents the bootstrapped 95% confidence interval for the true-positive rate at each false-positive rate. An asterisk marks the point corresponding to the optimal PC1 threshold depicted in B. FIG. 7D shows PCA loading plot depicting the relative contributions of each bile acid to the distribution of Cx⁻/EIA⁻ samples in A. Abbreviations are indicated in Table 3.

FIG. 8A-8D show a principal component analysis of GC-MS-defined metabolome in the clinical cohort. FIG. 8A is a PCA plot of uninfected patients' GC-MS metabolomes (green, n=62), onto which is projected the GC-MS metabolomes of patients with CDI (red, n=62). Data ellipses are drawn around each group of samples (95% level). The clustering of CDI specimens at high PC1 values is consistent with a favored metabolomic profile among patients with CDI. FIG. 8B is a dot plot of PC1 scores for each patient (n=62 in each group). Gray dashed line depicts the PC1 threshold that maximizes the sum of percent sensitivity and specificity for distinguishing Cx⁻/EIA⁻ from Cx⁺/EIA⁺ samples. FIG. 8C is an ROC plot evaluating the ability of PC1 to distinguish between CDI patients and controls. The gray region represents 95% confidence intervals bootstrapped for the true-positive rate at each possible false-positive rate. An asterisk marks the point corresponding to the optimal PC1 threshold depicted in panel B. FIG. 8D is a plot of PC1 and PC2 loadings for all 2539 GC-MS features. It depicts the relative contributions of each GC-MS feature to the distribution of Cx⁻/EIA⁻ samples in the PCA projection in A. Features in the top or bottom 1% of PC1 loadings tentatively identified as sugars or sugar alcohols are highlighted in blue.

FIG. 9A-9D show the interrelationships between host and C. difficile-associated metabolites. FIG. 9A shows plotting bile acid PC1 (FIG. 7) versus 4-methylpentanoic acid index (FIG. 4) reveals that high PC1 score and high 4-methylpentanoic acid index values coincide in patients with CDI compared with control patients (n=32 for each group). The dashed line marks the dividing line assigned 50% probability of being Cx⁺/EIA⁺ by a logistic regression model incorporating both PC1 and 4-methylpentanoic acid index. FIG. 9B probabilities assigned to each patient by the logistic regression model (n=32 per group). Higher values indicate higher certainty of Cx⁺/EIA⁺ status. The gray line marks the 50% probability cutoff above which samples are considered Cx⁺/EIA⁺. FIG. 9C is a ROC curve showing the performance of the logistic regression model in discriminating Cx⁻/EIA⁻ patients from Cx⁺/EIA⁺ patients. The gray region represents 95% confidence intervals bootstrapped for the true-positive rate at each possible false-positive rate. The AUC and its 95% confidence interval are also reported.

FIG. 9D is a Euler diagram showing the overlap between culture, EIA, and metabolome status. Samples were considered metabolome-positive if assigned a probability above 50% by the logistic regression model.

FIG. 10A-10B show cohort assembly from clinical stool specimens. FIG. 10A shows a flow chart for selection of Cx−/EIA− and Cx+/EIA− specimens. FIG. 10B shows a flow chart for selection of Cx+/EIA+ specimens.

FIG. 11A-11R shows GC-MS spectra for each detected Stickland metabolite (red) compared to the standard spectra in the NIST 14 library. Sample spectra were obtained from a pooled quality control sample using the AMDIS GC-MS software package. FIG. 11A shows the GC-MS spectra for leucine. FIG. 11B shows the GC-MS spectra for 4-MPA. FIG. 11C shows the GC-MS spectra for 2-OH-4-MPA. FIG. 11D shows the GC-MS spectra for isoleucine. FIG. 11E shows the GC-MS spectra for 2-OH-3-MPA.

FIG. 11F shows the GC-MS spectra for valine. FIG. 11G shows the GC-MS spectra for 2-OH-3-MBA. FIG. 11H shows the GC-MS spectra for phenylalanine. FIG. 11I shows the GC-MS spectra for PAA. FIG. 11J shows the GC-MS spectra for PPA. FIG. 11K shows the GC-MS spectra for tyrosine. FIG. 11L shows the GC-MS spectra for 4-OH-PAA. FIG. 11M shows the GC-MS spectra for 3-4-OH-PPA. FIG. 11N shows the GC-MS spectra for ornithine. FIG. 11O shows the GC-MS spectra for proline. FIG. 11P shows the GC-MS spectra for 5-APA. FIG. 11Q shows the GC-MS spectra for tryptophan. FIG. 11R shows the GC-MS spectra for IAA.

FIG. 12 shows a selected ion (m/z 158) GC-MS chromatogram at from a Cx+/EIA+ specimen showing a peak (arrow) associated with Cx+/EIA+ specimens in multivariate analyses. Both this and the immediately subsequent peak at 8.4 min (identified as isoleucine) possess similar EI mass spectra and match the NIST14 Library for isoleucine. The peak at 8.1 min corresponds to isoleucine based on comparison to an authentic standard. Among the metabolites associated with Cx+/EIA+ were two closely eluting peaks with mass spectra matching that of isoleucine. were two chromatographically-distinct molecules with mass spectra matching isoleucine. The first of these metabolites was positively-associated with CDI. With this GC-MS method, differing peaks with identical mass spectra can result from the presence of isomers (but not enantiomers). When we compared these peaks with authentic leucine, tertiary leucine, and allo-isoleucine, the first peak matched L-leucine and the second peak matched allo-isoleucine.

FIG. 13 shows a selected ion (m/z 158) GC-MS chromatogram of isoleucine isomers. Of these, allo-isoleucine yields a peak that elutes immediately prior to isoleucine, possesses a similar mass spectrum, and corresponds to the Cx⁺/EIA⁺-associated peak noted above in FIG. 12.

FIG. 14A-14C show the product/precursor ratios of several Stickland metabolites monitored by SIM. FIG. 14A show structures of Stickland precursors and products. FIG. 14B show a dot plots of product/precursor ratios for all three patient groups. FIG. 14C show ROC plots showing true- and false-positive rates at varying threshold product/precursor ratios. The area under the curves and 95% confidence intervals are displayed on the plots.

FIG. 15 shows a GC-MS chromatogram of authentic isodeoxycholic acid (iso-DCA), isolithocholic acid (iso-LCA), lithocholic acid (LCA), deoxycholic acid (DCA), cholic acid (CA), chenodeoxycholic acid (CDCA), hyodeoxycholic acid (HDCA), ursodeoxycholic acid (UDCA), or 7-ketodeoxycholic acid (7-keto-DCA).

FIG. 16 shows EI-MS spectra of the fecal metabolite BA1 (top) and authentic lithocholic acid (bottom). Molecular ions are indicated in the red boxes. RT: gas chromatographic retention time.

FIG. 17 shows EI-MS spectra of the fecal metabolite BA1 (top) and authentic cholanic acid (bottom).

FIG. 18 shows GC-MS selected ion chromatogram at m/z 430 of fecal specimen showing BA1 with closely-eluting peak (delta-2) (top panel). EI mass spectra of BA1 (middle panel) and delta-2 (bottom panel). Red arrows denote peaks with quantitative differences between the two features.

FIG. 19 shows proposed, favored retro-Diels-Alder fragmentation mechanism for delta-2-cholenoic acid.

FIG. 20 shows EI-MS spectra of the fecal metabolite BA2 (top) and authentic deoxycholic acid (bottom). Molecular ions are indicated in the red boxes. RT, gas chromatographic retention time.

FIG. 21 shows a product ion spectrum of LCA-S in a representative patient sample. The LCA-S spectrum exhibits a 96.9 m/z fragment characteristic of sulfated bile acids.

FIG. 22 shows precursor ion spectrum of LCA-S standard from its aliphatic sulfate product ion (96.7 m/z).

FIG. 23 shows precursor ion spectra of bile acid sulfates in a representative patient sample. Shaded boxes in top panel are time periods from which the corresponding spectra were extracted in subsequent panels.

FIG. 24A-24B show selected ion monitoring (SIM) GC-MS chromatograms of fecal specimens containing ¹³C6-trehalose internal standard and monitoring for peaks corresponding to trehalose. Within each panel, the top chromatogram is the monitored ion for trehalose at m/z 361 and the bottom chromatogram is the ion for ¹³C6-trehalose at m/z 367. FIG. 24A is a GC-MS chromatograms from patient fecal specimen in which trehalose was detected. This is indicated by the prominent, co-eluting peaks at 22.8 minutes. This specimen was determined to possess 230 pmol of trehalose. A comparable m/z 361 peak at this retention time was observed in the full scan metabolomic profiling data for this specimen.

FIG. 24B show GC-MS chromatograms from patient fecal specimen in which trehalose was undetectable. The m/z 361 chromatogram does not possess a co-eluting peak at 22.8 minutes that exceeds the background level.

FIG. 25 shows Putative identities of the upper and lower 1% of features in GC-MS PC1 from FIG. 8D. Peak spectra were matched against the NIST 14 spectral library. Features without a database match are labeled as “most intense fragment @ retention time in minutes.

FIG. 26 shows bile acid PC1 values (FIG. 7) plotted against GC-MS PC1 values (FIG. 8) for all Cx−/EIA− fecal specimens (n=62). Grey line represents least-squares regression line fitted to all data points (r2=0.007).

FIG. 27 shows amino acids detected by GC-MS in patient samples. Bars represent mean peak intensity and errors represent SEM. Significant differences between groups were identified using the Mann-Whitney U-test (**p<0.01, *p<0.05).

### DETAILED DESCRIPTION

Each year in the United States, over 450,000 cases of Clostridioides difficile infection (CDI) are associated with over 29,000 associated deaths, with attributable costs of over $2 billion (Lessa F C, et al., N Engl J Med. 2015; 372(9):825-834). CDI is the most common healthcare-associated infection in US hospitals, and most cases start outside of the hospital setting (Magill S S, et al., N Engl J Med. 2018; 379(18):1732-1744). Although antimicrobial exposures are clearly a critical CDI risk factor, the mechanisms contributing to this association are incompletely understood.

Given the risk for antimicrobial resistant (AMR)-pathogens causing life-threating infections, successful infectious disease management is critically dependent on identifying the most susceptible patient and determining the antibiotic susceptibility of the offending pathogen(s) to facilitate rapid clinical intervention. Although the value of precision infection management is well-recognized within the infectious disease community, neither the current analytical technology nor our understanding of host-pathogen risk associations is sufficiently well developed to initiate effective implementation.

The present disclosure is directed to methods and compositions that provide for accurate detection of C. difficile infection (CDI) in an individual. The methods can determine if an individual has CDI or does not have CDI. The methods can determine if an individual is at risk for CDI or is not at risk for CDI. Embodiments of the disclosure provide methods of identifying individuals that have CDI or are at risk for CDI (compared to a reference population) and identifying individuals that do not have CDI or are not at risk for CDI (compared to a reference population). The methods include determining analyte signatures as identified and reported herein. Such analyte signatures can be relied on to determine suitable treatment or adjust current therapy for subjects who need the treatment.

Additional aspects of the disclosure are described below.

### (I) Methods

One aspect of the present disclosure relates to methods for identifying a subject having or at risk for CDI (e.g., whether the subject has active disease), based on the analyte signature as disclosed herein. An analyte, as used herein, refers to a substance in a biological sample that may be measured as an indication of the health/disease state of a subject. As used herein, “analyte” is equivalent to “feature” or “biomarker.” For instance, an analyte may be a protein (e.g. a chemokine, toxin, an antibody, or other protein), an amino acid, a fatty acid (e.g. a short chain fatty acid), a bile acid, a carbohydrate or carbohydrate moiety (e.g. a sugar, a starch, or a proteoglycan), a lipid or lipid moiety, a nucleotide or nucleotide sequence, or other biomolecule. An analyte may be extracellular, or the biological sample may be treated so as to release an intracellular analyte using means known in the art. Alternatively, an analyte may be present on the surface of a cell in a biological sample. Such samples may be treated so as to release the analyte from the cell membrane or cell surface using means known in the art.

The terms “Clostridioides difficile infection”, “C. difficile infection” or “CDI” as used herein refers to an individual that has presence of Clostridioides difficile in their body to an extent and under conditions in which a sufficient level of toxins from the Clostridioides difficile results in symptoms such as diarrhea, abdominal pain, megacolon, or pseudomembranous colitis. This is in contrast to presence of Clostridioides difficile in an individual that is considered a carrier for the bacteria (e.g. a subject colonized with C. difficile) and that has no symptoms.

### (a) Analyte Signatures

An analyte signature refers to a characteristic expression profile of a single or a group of analytes that is indicative of an altered or unaltered biological process, medical condition, or a subject's responsiveness/non-responsiveness to a specific therapy. The analyte signatures disclosed herein encompass characteristic profiles of at least one analyte selected from short chain fatty acid series, amino acid metabolism, bile acids, carbohydrates, lipids, alcohols, toxins, and microbial nucleic acids which are identified as differentially expressed in a biological sample obtained from a subject relative to a reference value. See, e.g., the Examples below. In various embodiments, determining analyte levels can be supplemented with diagnostic assays such as assays to determine presence, absence, and/or quantity of a pathogen, clinical assays (e.g., those described in the below examples), advanced radiographic assays, diagnostic assays (e.g., PCR and/or ELISA) and aspiration.

In some embodiments, the present disclosure provides methods of detecting CDI in a subject. Additionally disclosed are methods of distinguishing a subject with an active CDI infection versus a C. difficile colonized, asymptomatic subject.

The analyte signatures as disclosed herein may represent the expression profile of at least one analyte, for example, at least 2 analytes, at least 3 analytes, at least 4 analytes, at least 5 analytes, at least 6 analytes, at least 7 analytes, at least 8 analytes, at least 9 analytes, at least 10 analytes, at least 15 analytes, at least 20 analytes, at least 25 analytes, at least 30 analytes, at least 35 analytes, at least 40 analytes, at least 45 analytes, at least 50 analytes or more. In some examples, the analyte signature may comprise multiple analytes with increased levels relative to a reference value. In other examples, the analyte signature may comprise multiple analytes with decreased levels relative to a reference value. In yet other examples, the analyte signature may comprise both increased and decreased analyte levels relative to a reference value.

In some embodiments, the analyte signature may comprise multiple analytes involved in multiple biological pathways, for example, 2 biological pathways, 3 biological pathways, 4 biological pathways, 5 biological pathways, 6 biological pathways, 7 biological pathways, 8 biological pathways, 9 biological pathways, 10 biological pathways, 11 biological pathways, 12 biological pathways, 13 biological pathways, 14 biological pathways, or 15 biological pathways. In some embodiments, the analyte signature comprises one or more analytes from an amino acid metabolism pathway (e.g. amino acid fermentation). In some embodiments, the analyte signature comprises one or more analytes from bile acid metabolism pathway. In some embodiments, the analyte signature comprises one or more analytes from carbohydrate metabolic pathway. In some embodiments, the analyte signature comprises one or more analytes from lipid metabolic pathway. In some embodiments, the analyte signature comprises one or more analytes from a biological pathway comprising a compound of Table 6 and/or Table 7 as a component of the biological pathway.

In some examples, the analyte signature comprises at least one short chain fatty acid. Non-limiting examples of short chain fatty acids to be used as an analyte in the methods described herein include 4-methylpentanoic acid (4-MPA), 2-hydroxy-4-methylpentanoic acid, 2-hydroxy-3-methylpentanoic acid, 2-hydroxy-3-methylbutanoic acid, phenylpropanoic acid, 3-(4-hydroxyphenyl)-propanoic acid, 5-aminopentanoic acid. In specific examples, the short chain fatty acid analyte(s) include 4-methylpentanoic acid, 2-hydroxy-4-methylpentanoic acid, 2-hydroxy-3-methylbutyric acid, 3-(4-hydroxyphenyl)propanoic acid (phloretic acid), or a combination thereof. In some embodiments, the short chain fatty acid analyte is a short chain fatty acid as described in Table 6, Table 7, and/or the below Examples. In some embodiments, the analyte is determined by quantifying short chain fatty acid activity through product/precursor ratios. In a non-limiting example, leucine is precursor to 4-MPA and therefore an analyte useful in the present methods is the 4-MPA/leucine ratio.

In some examples, the analyte signature comprises at least one amino acid. Non-limiting examples of amino acids to be used as an analyte in the methods described herein include isoleucine, allo-isoleucine, leucine, proline, phenylalanine, ornithine and valine. In some embodiments, the analyte signature comprises at least one branched chain amino acid. In some embodiments, the analyte is determined by quantifying amino acid through product/precursor ratios. In a non-limiting example, isoleucine is precursor to allo-isoleucine and therefore an analyte useful in the present methods is the allo-isoleucine/isoleucine ratio. In some embodiments, the amino acid analyte is an amino acid as described in Table 6, Table 7, and/or the below Examples.

In some examples, the analyte signature comprises at least one analyte that is involved in bile acid metabolism. Non-limiting examples of analytes to be used in the methods described herein include cholenoic acid, hydroxycholenic acid, deoxycholic acid, chenodeoxycholic acid, lithcholic acid. In some examples, the analyte involved in bile acid metabolism is a noncanonical unsaturated, dehydroxylated bile acid. In some embodiments, the analyte that is involved in bile acid metabolism comprise canonical primary, secondary bile acids, classic primary bile acids, glycine or taurine conjugates, conjugated secondary (dehydroxylated) bile acids, secondary bile acid sulfates, di-hydroxylated cholenic acid sulfate, sulfated bile acids, sulfated cholenic acid, dehydroxylated cholenic acid sulfates including sulfated keto bile acids and sulfated secondary bile acids. In some embodiments, the analyte that is involved in bile acid metabolism are one or more of cholic, chenodeoxycholic, deoxycholic and lithocholic acids. In some embodiments, the bile acid analyte is a bile acid as described in Table 6, Table 7, and/or the below Examples. In some embodiments, the bile acid analyte is a modified bile acid-to-an unmodified bile acid ratio.

In some examples, the analyte signature comprises at least one analyte that is involved in carbohydrate metabolism. Non-limiting examples of analytes to be used in the methods described herein include monosaccharides, disaccharides, and sugar alcohols. In some examples, the analyte involved in carbohydrate metabolism is one or more of fructose, ribitol, and/or glyceryl glycoside. In some embodiments, the carbohydrate analyte is a bile acid as described in Table 6, Table 7, and/or the below Examples.

In some embodiments, the analyte signature comprises at least one analyte and/or analyte from a corresponding biological pathway as described in Table 6, Table 7, and/or the below Examples.

### (b) Determination of Analyte Signatures

To determining any of the analyte signatures as disclosed herein, analyte levels in a biological sample of a candidate subject can be measured by routine practice. In particular embodiments, the one or more analytes are in the form of protein, and assays are performed to measure the level of the respective protein(s). A particular protein feature may be analyzed solely for a method, or multiple proteins may be analyzed either separately or simultaneously. Protein features may originate from the host or from a microbe in the host. Protein detection methods may utilize spectrometry methods (such as high performance liquid chromatography or mass spectrometry) or antibody-based methods, such as enzyme-linked immunosorbent assays (ELISA) or western blot. The term “antibody” is used to refer to any antibody-like molecule that has an antigen binding region, and includes antibody fragments such as Fab′, Fab, F(ab′) 2, single domain antibodies (DABs), Fv, scFv (single chain Fv), and the like. In specific embodiments, metabolites are analyzed by mass spectrometry, ELISA, chromatography, or a combination thereof, and analytes are analyzed by mass spectrometry, ELISA, chromatography, Western blotting, immunoprecipitation, Immunoelectrophoresis, or a combination thereof. In specific embodiments, the analytes are detected by mass spectrometry. Mass spectrometry methods may include MALDI-TOF, LC-MS, GC-MS, IC-MS, for example. See, e.g., Molecular Cloning: A Laboratory Manual, J. Sambrook, et al., eds., Third Edition, Cold Spring Harbor Laboratory Press, Cold Spring Harbor, N.Y., 2001, Current Protocols in Molecular Biology, F. M. Ausubel, et al., eds., John Wiley & Sons, Inc., New York. Microarray technology is described in Microarray Methods and Protocols, R. Matson, CRC Press, 2009, or Current Protocols in Molecular Biology, F. M. Ausubel, et al., eds., John Wiley & Sons, Inc., New York.

Clostridioides difficile, a common nosocomial pathogen, has been listed as top urgent threat to public health by CDC. C. difficile infection (CDI) after antibiotic therapy is effectively cured by fecal microbiota transplantation (FMT) by restoring heathy gut microbiota. Medications and therapy that disrupt gut microbiota are well recognized CDI risk factors supporting the concept that microbiota health is a key determinant in patient susceptibility to C. difficile. Although testing for microbiota susceptibility to CDI is evolving, it is still poorly developed. Embodiments of the disclosure provide methods and compositions related to guidelines for suitability of treatment for Clostridioides difficile infection.

A subject to be assessed by any of the methods described herein can be a mammal, e.g., a human subject having CDI. A subject having CDI may be diagnosed based on clinically available tests and/or an assessment of the pattern of symptoms in a subject and response to therapy. In some embodiments, the subject is a pediatric subject. A pediatric subject may be of 18 years old or below. In some examples, a pediatric patient may have an age range of 0-12 years, e.g., 6 months to 8 years old or 1-6 years. In some instances, the subject may be free of a prior treatment for microbial infection, for example, free of any antibiotic or anti-viral treatment. In some instances, the subject may have received a prior treatment for a microbial infection. The individual may be of any kind, and the methods may be performed before, during, or after the individual has a symptom of CDI. The methods may be performed when the individual is in need of antibiotics and/or antimicrobials of any kind or when the individual has already had antibiotics and/or antimicrobials of any kind. The methods may be performed as routine medical practice for an individual.

In some embodiments, the individual may or may not be a carrier of C. difficile. Individuals that are carriers of C. difficile would score positively for standard CDI assays (such as with 16S ribosomal RNA (rRNA)), but in methods of the disclosure they may be subjected to method steps that allow for determination of a cause of symptom associated with CDI. The individual may be of an age in which the individual is not responsive to C. difficile toxins, and that individual may be assayed for and, in some cases, may be determined to have, diarrhea from a cause other than CDI. An individual may mature to the point that they become susceptible to CDI, and beyond that stage the individual may be subjected to methods encompassed herein to determine whether or not their diarrhea is from CDI. In some embodiments, adults are subjected to methods of the disclosure to determine whether or not they have CDI. Adults generally are low risk for CDI unless they have taken an antibiotic and/or antimicrobial, including taken any antibiotic and/or antimicrobial at any time in their life or taken any antibiotic and/or antimicrobial within a certain time frame, such as within 10, 9, 8, 7, 6, 5, 4, 3, or 2 years, or within 1 year, or within 12, 11, 10, 9, 8, 7, 6, 5, 4, 3, or 2 months, or within 1 month, or within 4, 3, or 2 weeks, or within 1 week. An individual that has taken an antibiotic and/or antimicrobial is at greater risk for CDI than an individual that has not taken an antibiotic and/or antimicrobial, and this historical information for the individual may or may not be considered in determination of an outcome.

In some embodiments, an individual may not have a symptom of CDI but is still subjected to analysis methods encompassed herein to determine an increased risk for having CDI. Any individual that is considered high risk for CDI may be provided a suitable treatment to prevent CDI, such as one or more antibiotics or prophylactic therapy including anti-virulence and/or microbial therapy. An individual may be determined to be a high risk individual based on the outcome of methods performed herein based on their genotype, family history, personal history, and overall health, including whether or not they already have a medical condition that may or may not be pathogenic infection and/or may or may not have diarrhea as a symptom. For example, an individual with a particular medical condition may be at high risk, moderate risk, or low risk for CDI. In one embodiment, an individual is high risk for CDI if they already have or have had antibiotic-associated diarrhea, acute myeloid leukemia, allogeneic hematopoietic stem cell transplantation, or have been in or are in an intensive care unit of a medical facility. Such an individual may or may not be provided a CDI treatment or prophylaxis. In another embodiment, an individual may be moderate risk for CDI if they already have or have had inflammatory bowel disease or cirrhosis. In a particular embodiment, an individual may be at low risk for CDI if they have or have had functional gastrointestinal disorders, metabolic syndrome, rheumatoid arthritis, or atherosclerosis.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

As used herein, the term “biological sample” refers to a sample obtained from a subject. A suitable biological sample can be obtained from a subject as described herein via routine practice. Non-limiting examples of biological samples include fluid samples such as blood (e.g., whole blood, plasma, or serum), urine, synovial fluid, and saliva, and solid samples such as tissue (e.g., skin, lung, or nasal) and feces. Such samples may be collected using any method known in the art or described herein, e.g., buccal swab, nasal swab, venipuncture, biopsy, urine collection, or stool collection. In some embodiments, the biological sample can be a fecal sample.

In some embodiments, a single sample is obtained from a subject to detect the analyte signature in the sample. Alternatively, the analyte signature may be detected in samples obtained over time from a subject. As such, more than one sample may be collected from a subject over time. For instance, 2, 3, 4, 5, 6, 7, 8, 9, 10, 11, 12, 13, 14, 15, 16 or more samples may be collected from a subject over time. In some embodiments, 2, 3, 4, 5, or 6 samples are collected from a subject over time. In other embodiments, 6, 7, 8, 9, or 10 samples are collected from a subject over time. In yet other embodiments, 10, 11, 12, 13, or 14 samples are collected from a subject over time. In other embodiments, 14, 15, 16 or more samples are collected from a subject over time.

When more than one sample is collected from a subject over time, samples may be collected every 0.5, 1, 2, 3, 4, 5, 6, 7, 8, 9, 10, 11, 12 or more hours. In some embodiments, samples are collected every 0.5, 1, 2, 3, or 4 hours. In other embodiments, samples are collected every 4, 5, 6, or 7 hours. In yet other embodiments, samples are collected every 7, 8, 9, or 10 hours. In other embodiments, samples are collected every 10, 11, 12 or more hours. Additionally, samples may be collected every 1, 2, 3, 4, 5, 6, 7, 8, 9, 10, 11, 12 or more days. In some embodiments, a sample is collected about every 6 days. In some embodiments, samples are collected every 1, 2, 3, 4, or 5 days. In other embodiments, samples are collected every 5, 6, 7, 8, or 9 days. In yet other embodiments, samples are collected every 9, 10, 11, 12 or more days.

In some embodiments, once a sample is obtained, it is processed in vitro to detect and measure the amount of the analyte signature. All suitable methods for detecting and measuring an amount of a protein or protein product thereof known to one of skill in the art are contemplated within the scope of the invention. For example, epitope binding agent assays (i.e. antibody assays), enzymatic assays, electrophoresis, chromatography and/or mass spectrometry may be used. Non-limiting examples of epitope binding agent assays include an ELISA, a lateral flow assay, a sandwich immunoassay, a radioimmunoassay, an immunoblot or Western blot, flow cytometry, immunohistochemistry, and an array. The expression level(s) of the proteins involved in any of the protein signature as disclosed herein may be represented by the level of the mRNAs. Methods for detecting and/or assessing a level of nucleic acid expression in a sample are well known in the art, and all suitable methods for detecting and/or assessing an amount of nucleic acid expression known to one of skill in the art are contemplated within the scope of the invention. Non-limiting examples of suitable methods to assess an amount of nucleic acid expression may include arrays, such as microarrays, PCR, such as RT-PCR (including quantitative RT-PCR), nuclease protection assays and Northern blot analyses.

The level of the target analyte may be normalized to the level of a control analyte. This allows comparisons between assays that are performed on different occasions. For example, the raw data of analyte levels can be normalized against the expression level of an internal control, e.g. RNA (e.g., a ribosomal RNA or U6 RNA). The normalized expression level(s) of the analytes can then be compared to the level(s) of the same analytes of a control sample, which can be normalized against the same internal control, to determine whether the subject is likely to have an active CDI or responsive to a therapeutic treatment or non-responsive to a therapeutic treatment.

Based on the levels of the analytes disclosed herein, an analyte signature can be obtained via, e.g., a computational program. Various computational programs can be applied in the methods of this disclosure to aid in analysis of the expression data for producing the analyte signature. Examples include, but are not limited to, Prediction Analysis of Microarray (PAM; see Tibshirani et al., PNAS 99(10):6567-6572, 2002); Plausible Neural Network (PNN; see, e.g., U.S. Pat. No. 7,287,014), PNNSulotion software and others provided by PNN Technologies Inc., Woodbridge, Va., USA, and Significance Analysis of Microarray (SAM). In some examples, a analyte signature may be represented by a score that characterizes the expression pattern of the analytes involved in the analyte signature.

(c) Assessing Disease Occurrence and/or Severity and Therapeutic Responsiveness Based on Analyte Signature and Optionally Other Factors

In an aspect, the disclosure provides a method to identify a subject having CDI based on the analyte profiles described herein measured in a biological sample obtained from the subject. The method generally comprises (i) measuring the expression level of at least one analyte, in the biological sample, (ii) determining if the analyte level is increased or decreased relative to a reference value, and (iii) identifying the subject as having or not having CDI. In some embodiments, the subject is classified as having CDI if the expression level of one or more of short chain fatty acids, allo-isoleucine, 4-MPA are increased relative to the reference value and/or if the expression level of one or more amino acids, secondary bile acids, cholenoic acid, monohydroxycholenoic acid, deoxycholic acid, lithocholic acid, fructose are decreased relative to the reference value. In some embodiments, the subject is classified as not having CDI if the expression levels are the same relative to the reference value. In some embodiments, the subject is classified as having or not having CDI or responsive or non-responsive to therapy by using an analyte signature as provided in Table 6, Table 7 and/or the below Examples.

In still yet another aspect, the disclosure provides a method for monitoring CDI in a subject. In such an embodiment, a method of detecting the analyte level may be used to assess disease severity of a subject at one point in time. Then at a later time, the method of detecting the analyte level of the CDI signature may be used to determine the change in disease severity of the subject over time. For example, the method of detecting the analyte level of expression of the CDI signature may be used on the same subject days, weeks, months or years following the initial determination of the analyte level CDI signature. Accordingly, the method of detecting the analyte level of CDI signature may be used to follow a subject over time to determine the rate of disease progression. For example, if the analyte level of the CDI signature is changed relative to the analyte level of the CDI signature obtained from the same subject at an earlier time point, may indicate an abatement of disease progression. Alternatively, if the analyte level of the CDI signature similar relative to the analyte level of the CDI signature from the same subject at an earlier time point, may indicate disease progression.

Any of the analyte signatures of a candidate subject as disclosed herein can be used for assessing whether the subject's responsiveness or non-responsiveness to a therapy, for example, an antibiotic or CDI-therapy. For example, the analyte signature of a candidate subject can be compared with a reference value. As used herein, assessing “responsiveness” or “non-responsiveness” to a therapeutic agent refers to the determination of the likelihood of a subject for responding or not responding to the therapeutic agent.

A reference value may represent the same analyte signature of a control subject or represent the same analyte signature of a control population. In some examples, the same analyte signature of a control subject or a control population may be determined by the same method as used for determining the analyte signature of the candidate subject. In some instances, the control subject or control population may refer to a healthy subject or healthy subject population of the same species (e.g., a human subject or human subject population having no disease). Alternatively, the control subject or control population may be a CDI patient or CDI patient population who are responsive to any of the therapeutic agents disclosed herein or known in the art to treat CDI. In other instances, the control subject or control population may be a CDI patient or CDI patient population who is non-responsive to the therapeutic agent.

It is to be understood that the methods provided herein do not require that a reference value be measured every time a candidate subject is tested. Rather, in some embodiments, it is contemplated that the reference value can be obtained and recorded and that any test level can be compared to such a reference level. The reference level may be a single-cutoff value or a range of values.

By comparing the analyte signature of a candidate subject as disclosed herein and a reference value as also described herein, the subject can be identified as responsive or likely to be responsive or as not responsive or not likely to be responsive to treatment based on the assessing.

For example, when the reference value represents the same analyte signature of subjects who are responsive to a therapy, derivation from such a refer value would indicate non-responsiveness to the therapy. Alternatively, when the reference value represents the same analyte signature of patients who are non-responsive to a therapy, derivation from such a reference value would indicate responsiveness to the therapy. In some instances, derivation means that the analyte signature (e.g., represented by a score) of a candidate subject is elevated or reduced as relative to a reference value, for example, by at least 5%, 10%, 20%, 30%, 40%, 50%, 60%, 70%, 80%, 90%, 100%, 150%, 200%, 300%, 400%, 500% or more above or below the reference value.

By comparing the CDI occurrence and/or disease severity analyte signature of a candidate subject as disclosed herein and a reference value as also described herein, the subject can be identified as having or at risk for the disease, or having active disease.

For example, when the reference value represents the same analyte signature of healthy controls, derivation from such a reference value would indicate disease occurrence of risk for the disease. Alternatively, when the reference value represents the same analyte signature of patients in inactive disease state, derivation from such a reference value would indicate active disease.

### (c) Therapeutic Application of Analyte Signatures

When a subject is determined to be responsive or non-responsive based on any of the analyte signatures disclosed herein, this subject could be subjected to a suitable treatment for CDI, including any of the CDI treatments known in the art and disclosed herein. Alternatively, when a subject is determined as having or at risk for CDI or having active disease based on any of analyte signatures as also disclosed herein, such a subject may be given a suitable antibiotic or antimicrobial therapy, for example, those described herein. Thus, as described herein, a subject having CDI can be treated by any method known in the art suitable for treating the disease. Therapeutic agents and methods of treating CDI are well known in the art.

For example, a therapeutic agent can be any agent suitable for treating CDI or any agent suitable to avoid CDI.

As another example, a therapeutic agent can be an antibiotic such as methicillin, glycopeptide, tetracycline, oxytetracycline, doxycycline; chlortetracycline, minocycline, glycylcycline, cephalosporin, ciprofloxacin, nitrofurantoin, trimethoprim-sulfa, piperacillin/tazobactam, moxifloxacin, vancomycin, teicoplanin, penicillin, and macrolide. As another example a therapeutic agent can be an antiviral agent (e.g., a broad-spectrum antiviral or viral specific antiviral), such as oseltamivir (Tamiflu), zanamivir (Relenza), and peramivir (Rapivab). As another example, a therapeutic agent can be an anti-inflammatory agent. Non-limiting examples of anti-inflammatory agents include sulfasalazine, mesalamine, balsalazide, olsalazine, or corticosteroids (e.g., prednisone or budesonide). In some embodiments, treatment comprises surgery. Non-limiting examples of surgery include fecal transplant.

The term “treating” as used herein refers to the application or administration of a composition including one or more active agents to a subject, who has CDI, or a predisposition toward CDI, with the purpose to cure, heal, alleviate, relieve, alter, remedy, ameliorate, improve, or affect the disease, the symptoms of the disease, or the predisposition toward the disease. An “effective amount” is that amount of an anti-CDI agent that alone, or together with further doses, produces the desired response, e.g. eliminate or alleviate symptoms, prevent or reduce the risk of flare-ups (maintain long-term remission), and/or restore quality of life. The desired response is to inhibit the progression of the disease. This may involve only slowing the progression of the disease temporarily, although more preferably, it involves halting the progression of the disease permanently. This can be monitored by routine methods or can be monitored according to diagnostic and prognostic methods discussed herein. The desired response to treatment of the disease or condition also can be delaying the onset or even preventing the onset of the disease or condition.

Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Any of the methods described herein can further comprise adjusting the CDI treatment performed to the subject based on the results obtained from the methods disclosed herein (e.g., based on analyte signatures disclosed herein). Adjusting treatment includes, but are not limited to, changing the dose and/or administration of the anti-CDI agent used in the current treatment, switching the current medication to a different anti-CDI agent, or applying a new CDI therapy to the subject, which can be either in combination with the current therapy or replacing the current therapy.

In some embodiments, the present disclosure provides a method for treating a subject (e.g., a human patient) having CDI, the method comprising administering an effective amount of an anti-CDI agent (e.g., those disclosed herein) to a subject who exhibits an analyte signature indicative of having an active CDI.

Generally, a safe and effective amount of a therapeutic agent is, for example, that amount that would cause the desired therapeutic effect in a subject while minimizing undesired side effects. In various embodiments, an effective amount of a therapeutic agent described herein can substantially inhibit infection, slow the progress of infection or limit the development of infection.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of a therapeutic agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit heart failure, coronary heart disease, or cardiac related death, slow the progress of heart failure or coronary heart disease or limit the development of heart failure or coronary heart disease.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are proteinrally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a therapeutic agent can occur as a single event or over a time course of treatment. For example, a therapeutic agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a cardiovascular disease, disorder, or condition.

A therapeutic agent can be administered simultaneously or sequentially with another agent, such as an antibiotic, an anti-inflammatory, or another agent. For example, a therapeutic agent can be administered simultaneously with another agent, such as an antibiotic or an anti-inflammatory. Simultaneous administration can occur through administration of separate compositions, each containing one or more of a therapeutic agent, an antibiotic, an anti-inflammatory, or another agent. Simultaneous administration can occur through administration of one composition containing two or more of a therapeutic agent, an antibiotic, an anti-inflammatory, or another agent. A therapeutic agent can be administered sequentially with an antibiotic, an anti-inflammatory, or another agent. For example, a therapeutic agent can be administered before or after administration of an antibiotic, an anti-inflammatory, or another agent.

### II. Kits

One can recognize that based on the methods described herein, detection reagents, kits, and/or systems can be utilized to detect the analytes related to the disease signature for diagnosing an individual (the detection either individually or in combination). The reagents can be combined into at least one of the established formats for kits and/or systems as known in the art. As used herein, the terms “kits” and “systems” refer to embodiments such as combinations of at least one nucleic acid detection reagent, at least one metabolite detection reagent, and/or at least one protein detection reagent. Non-limiting examples of nucleic acid reagents include at least one nucleic acid isolation reagent, at least one selective oligonucleotide probe, at least one sequencing reagent, and/or at least one PCR primer. Non-limiting examples of metabolite detection reagents include at least one metabolite extraction reagent, at least one enzyme capable of detecting specific metabolites, at least one chromatography reagent, and/or at least one mass spectrometry reagent. Non-limiting examples of protein detection reagents include at least one protein isolation reagent, at least one protein-specific antibody, at least one chromatography reagent, and/or at least one mass spectrometry reagent.

The kits could also contain other reagents, chemicals, buffers, enzymes, packages, containers, electronic hardware components, etc. The kits/systems could also contain packaged sets of PCR primers, oligonucleotides, arrays, beads, or other detection reagents. Any number of probes could be implemented for a detection array. In some embodiments, the detection reagents and/or the kits/systems are paired with chemiluminescent or fluorescent detection reagents.

Particular embodiments of kits/systems include the use of electronic hardware components, such as DNA chips or arrays, or microfluidic systems, for example. In some embodiments, the kit provides a platform for performing mass spectrometry on the sample to measure the features disclosed herein. Mass spectrometry methods may include MALDI-TOF, LC-MS, GC-MS, IC-MS, for example. In particular embodiments, the kit provides a platform for performing an enzyme-linked immunosorbent assay (ELISA) to measure the levels of classifiers disclosed herein in a sample. In specific embodiments, the kit also comprises one or more therapeutic or prophylactic interventions in the event the individual is determined to be in need of.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As used herein, the following definitions shall apply unless otherwise indicated. For purposes of this invention, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry,” Thomas Sorrell, University Science Books, Sausalito: 1999, and “March's Advanced Organic Chemistry,” 5^{th} Ed., Smith, M. B. and March, J., eds. John Wiley & Sons, New York: 2001, the entire contents of which are hereby incorporated by reference.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.). Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Metabolic Networks Connect Host-Microbiome Processes to Human Clostridiodies difficile Infections

Clostridioides difficile infections (CDIs) arise following ingestion of C. difficile spores, which germinate in the intestinal tract and give rise to metabolically active, Gram-positive rods that colonize the colon. These vegetative forms secrete toxins whose effects upon the colonic epithelium give rise to a spectrum of intestinal symptoms ranging from diarrhea to a life-threatening pseudomembranous colitis. C. difficile persist with assistance from extensive antibiotic resistance that enables proliferation in patients whose intestinal microbiomes have been altered by broad-spectrum antibiotic exposure. Individual differences in CDI susceptibility and severity are also substantial, such that some patients harboring C. difficile do not benefit from C. difficile-directed antibiotic therapies (McDonald L C, et al., Clin Infect Dis. 2018; 66(7):987-994; Kwon J H et al., J Clin Microbiol. 2017; 55(2):596-605). The mechanistic bases for these individual differences are poorly understood. Adaptations to different chemical environments in the intestine may markedly affect the pathogenic potential of C. difficile (Casadevall A et al., PLoS Pathog. 2011; 7(7):e1002136).

C. difficile is regarded as an opportunistic colonizer that is susceptible to suppression by healthy intestinal microbiomes. A number of candidate metabolic functions may contribute to this suppressive activity (McDonald L C, et al., Clin Infect Dis. 2018; 66(7):987-994). One such function is the ability of healthy microbiomes to convert primary bile acids (e.g., cholic and chenodeoxycholic acids), which have been shown to promote spore germination in vitro, to secondary bile acids (e.g., deoxycholic and lithocholic acids, respectively). Both gnotobiotic or antibiotic-exposed mice exhibit diminished secondary bile acid production, though it is unclear which bile acid changes have causative, in addition to correlative, relationships with CDI susceptibility in humans (Khoruts A et al., Nat Rev Gastroenterol Hepatol. 2016; 13(9):508-516; Palmieri L J, et al., Front Microbiol. 2018; 9:2849; Theriot C M et al. mSphere. 2016; 1(1):e00045-15; Thanissery R et al., Anaerobe. 2017; 45:86-100). Recent work in an ex vivo murine model shows that secondary bile acids inhibit C. difficile germination and growth, although these effects are partially strain-specific (Theriot C M, et al., mSphere. 2016; 1(1):e00045-15, Thanissery R, et al., Anaerobe. 2017; 45:86-100). Multiple bile acid transformation pathways are plausible in humans, who possess distinctive bile acids and microbiome compositions that may contribute to CDI risk in species-specific ways.

Current approaches to CDI diagnosis require compatible laboratory findings in the context of attributable clinical symptoms (e.g., diarrhea, abdominal pain, megacolon) (McDonald L C, et al., Clin Infect Dis. 2018; 66(7):987-994). Two laboratory diagnostic approaches currently predominate in US hospitals, one based on nucleic acid amplification-based identification of toxigenic C. difficile and the other based on enzyme immunoassay detection of C. difficile exotoxins. The relative merits of these approaches have been debated (Fang F C, et al., J Clin Microbiol. 2017; 55(3):670-680). Direct detection of C. difficile (by culture or nucleic acid amplification) may be particularly susceptible to false-positive results for CDI due to detection of inactive spores, while there is concern that toxin detection tests are insufficiently sensitive and may yield false-negative results in some patients with CDI. Prospects for new approaches to improve diagnostic accuracy are therefore of interest.

To better understand the relationship between the intestinal metabolome and CDI in humans, the present example examines the fecal metabolomic profiling of hospitalized patients with diarrheal symptoms at an academic medical center. The cohort consists of patients with a toxigenic culture positive for C. difficile, either with or without a positive toxin enzyme immunoassay (EIA) result, alongside matched, uncolonized controls. To characterize fecal metabolomes, untargeted gas chromatography-mass spectrometry (GC-MS) was used, which permits robust chemical identification of metabolites and dietary compounds. Using multivariate analyses, multiple CDI-associated metabolites with microbial, host, and dietary origins were resolved. A distinctive short chain fatty acid (SCFA) series implicates extensive anaerobic amino acid metabolism by C. difficile in some colonized subjects. A novel, noncanonical bile acid correlation network not previously described in CDI susceptibility was also resolved. These and other results are consistent with numerous host-pathogen interactions that shape the relationship between patients and C. difficile. This allowed the assembly of a metabolomic definition of CDI from biochemical indices based on C. difficile-associated amino acid fermentation and host bile acid metabolism. These results can direct new CDI therapeutic and diagnostic efforts toward clinically relevant targets.

### Methods

Patient specimen collection. This cohort was derived from samples submitted for physician-ordered C. difficile toxin testing as part of routine clinical care. Remnant specimens that would have been otherwise discarded were frozen at −80° C. by the laboratory for future use. Approval was obtained from the Washington University Institutional Review Board with a waiver of informed consent to use specimens for this study. Patient and specimen evaluation for this cohort has recently been described (Dubberke E R, et al., Infect Control Hosp Epidemiol. 2018; 39(11):1330-1333). From August 2014 through September 2016, the Barnes-Jewish Hospital (BJH) Microbiology Laboratory detected the presence of toxin A and B in these specimens using the Alere TOX A/B II toxin enzyme immunoassay (EIA).

Inclusion and exclusion criteria. To identify and exclude patients with a potential alternate cause of diarrhea, BJH medical informatics databases were queried to identify patients with these conditions and medications. Patient charts lacking an identifiable alternate cause of diarrhea were reviewed to determine whether the patient had clinically significant diarrhea, and to confirm that there were no other known causes of diarrhea. If it was not possible to determine whether the patient had clinically significant diarrhea based on the medical records, the specimen was excluded. Specimens that were toxin negative (EIA⁻) were also excluded if the patient received treatment for CDI within 14 days of stool specimen collection. Due to these rigorous criteria, patients that were toxin positive (EIA⁺) were considered to have CDI and patients who were EIA⁻ but positive for a toxigenic strain of C. difficile (defined by the presence of tcdA and/or tcdB by PCR; Cx⁺) were considered colonized with toxigenic C. difficile but with diarrhea due to other reasons. EIA⁻ stools were also excluded if the patient was receiving antibiotics that could treat CDI to better ensure that patients with Cx⁺/EIA⁻ stool did not have CDI.

C. difficile culture and characterization. Briefly, 1 g stool was heat shocked at 80° C. for 10 minutes. The specimen was then placed into cycloserine, cefoxitin, mannitol broth with taurocholate and lysozyme (Anaerobe Systems) and incubated anaerobically at 35° C. When turbid, broth was streaked onto prereduced blood agar (BAP, Becton, Dickinson and Company). C. difficile was identified by matrix-assisted laser desorption/ionization time of flight (MALDI-TOF MS). Isolates were evaluated for the presence of tcdA, tcdB, and binary toxin genes (cdtA/cdtB) by multiplex PCR. PCR ribotyping was then performed. The ribotyping banding patterns were analyzed using DiversiLab Bacterial Barcodes software. Similarity of at least 95% was required for isolates to be considered identical. All unique strains were compared with the Cardiff-ECDC collection of C. difficile strains for name assignment. Isolates that did not match to a strain in the Cardiff-ECDC collection were compared with unique strains in the Washington University collection for name assignments. Isolates that did not match strains in the Cardiff-ECDC collection or Washington University collection were assigned a unique name.

Fecal extracts. Stool specimens were thawed on ice and approximately 0.1 mg of each was transferred to a microfuge tube and weighed. MeOH (1.25 mL, 70%) was added to each stool sample. The samples were sealed with parafilm, vortexed for 10 seconds, and rotated in a cold room for 2 hours. The samples were vortexed, decanted into a microcentrifuge tube and centrifuged at 20817×g in a desktop centrifuge for 15 minutes at 4° C. The supernatant was decanted into a tube and stored at −80° C. until analysis.

Gas chromatography-mass spectrometry (GC-MS). Stool extract (30 μL) was pipetted into a glass vial, dried under N₂, and derivatized with 100 μL MSTFA (N-Methyl-N-trimethylsilyltrifluoroacetamide)/CH3CN/pyridine (1:2.6:0.4), heated at 70° C. for 30 minutes, then cooled at room temperature overnight. Derivatized samples were analyzed using an Agilent 7890A gas chromatograph interfaced to an Agilent 5975C mass spectrometer and equipped with an HP-5MS column (30 m, 0.25 mm i.d., 0.25 μm film coating). For GC, an initial temperature of 80° C. for 2 minutes was followed by a linear gradient to 300° C. at 10° C./minute followed by a 5-minute elution at 300° C. EI was conducted with source temperature, electron energy, and emission current of 250° C., 70 eV, and 300 μA, respectively. The injector and transfer line temperatures were 250° C. For metabolite profiling and spectral analysis, the quadrupole was scanned from 50 to 650 m/z units. Structural information about GC-EI-MS features was obtained through spectral matching with the NIST 14 spectral library.

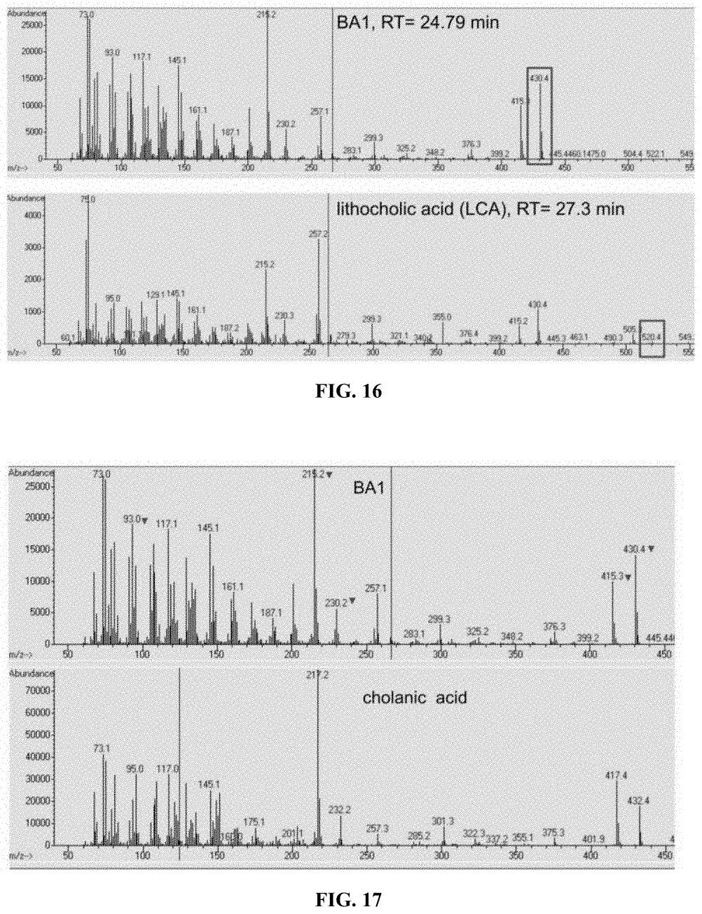

For targeted analyses of specific metabolites, the mass spectrometer monitored specific diagnostic ions for each compound. Each targeted metabolite was quantified in the selected ion monitoring mode in which ion chromatogram peak areas were determined at their corresponding retention times (Tables 1 and 2). For trehalose, stable isotope-labeled ¹³C-trehalose was added to each specimen as an internal standard before derivatization (Sergin I, et al., Nat Commun. 2017-8-15750). The peak areas of trehalose and ¹³ internal standards were calculated as a ratio (FIG. 16).

TABLE 1
Metabolites Quantified by Selective Ion Monitoring (SIM)

| | | GC retention time |
|---|---|---|
| Compound | m/z | (min) |
| 4-methylpentanoic acid | 173 | 4.73 |
| (4MP, isocaproic acid) | | |
| 2-hydroxy, 4-methylpentanoic acid | 159 | 7.54 |
| (2OH-4MP) | | |
| 2-hydroxy, 3-methylpentanoic acid | 159 | 7.59 |
| (2OH-3MP) | | |
| 2-(4-hydroxyphenyl)acetic acid | 179 | 12.8 |
| 3-(4-hydroxyphenyl)propionic acid | 179 | 14.1 |
| (phoretic acid) | | |
| allo-isoleucine | 158 | 8.37 |
| Isoleucine | 158 | 8.4 |
| Leucine | 158 | 8.1 |
| Tyrosine | 218 | 16.1 |
| cholic acid (CA) | 253 | 27.5 |
| cholenoic acid (CE) | 215, 430 | 24.7 |
| Trehalose | 361 | 22.8 |
| ¹³C-trehalose | 367 | 22.8 |

TABLE 2
Identification Bile Acids Among Cross-Validated CDI Features

| | | Base peak | |
|---|---|---|---|
| Feature | Retention time | (m/z) | Feature Identification |
| BA1 | 24.8 min | 215 | delta-2-cholenoic acid |
| BA2 | 25.44 min | 255 | hydroxycholenoic acid |
| BA3 | 27.44 min | 255 | deoxycholic acid (DCA) |

Measurement of bile acids by liquid chromatography-mass spectrometry. LC-ESI-MS/MS detection of each bile acid in fecal specimens or reference standards was performed with a Shimadzu UFLC coupled to a BetaSil C18 HPLC column (50 mm×2.1 mm×3 μm; Thermo Fisher Scientific) and an AB Sciex API 4000 QTrap mass spectrometer (AB Sciex) running in negative-ion electrospray ionization mode (ESI) using a Turbo V ESI ion source. Authentic bile acid standards (Table 3) were purchased and used to prepare 1 μM samples in 80% methanol. HPLC was conducted with a 0.4 mL/min flow rate using the following gradient: Solvent A (0.1% formic acid) and Solvent B (90% acetonitrile with 0.1% formic acid) were held constant at 95% and 5%, respectively, for 1 minute. Solvent B was increased to 98% by 8 minutes, held at 98% for 1 minute, and then reduced again to 5% in 1 minute. The column was equilibrated in 5% Solvent B for 3 minutes between runs. Optimized instrument settings are reported in Table 4.

TABLE 3
Fecal Bile Acids Monitored by LC-MS/MS

| | | | | Hydroxy- | Con- | | |
|---|---|---|---|---|---|---|---|
| Com- | | | Pri- | lations, | jugation | | Un- |
| munity^{a} | Abbrev. | Name^{b} | mary^{c} | No. | type | Sulfate | saturated |
| 1 | CA | Cholic acid | Yes | 3 | | | |
| | MCA | a-Muricholic acid | Yes | 3 | | | |
| | CDCA | Chenodeoxycholic acid | Yes | 2 | | | |
| | UDCA | Ursodeoxycholic acid | Yes | 2 | | | |
| 2 | G-CA | Glycocholic acid | Yes | 3 | Glyco | | |
| | T-CA | Taurocholic acid | Yes | 3 | Tauro | | |
| | HDCA | Hydeoxycholic acid | Yes | 2 | | | |
| | G-CDCA | Glycochenodeoxycholic acid | Yes | 2 | Glyco | | |
| | G-UDCA | Glycoursodeoxycholic acid | Yes | 2 | Glyco | | |
| | T-CDCA | Taurochenodeoxycholic acid | Yes | 2 | Tauro | | |
| | T-UDCA | Tauroursodeoxycholic acid | Yes | 2 | Tauro | | |
| 3 | G-DCA | Glycodeoxycholic acid | | 2 | Glyco | | |
| | T-DCA | Taurodeoxycholic acid | | 2 | Tauro | | |
| | T-LCA | Taurolithocholic acid | | 1 | Tauro | | |
| | G-LCA | Glycolithocholic acid | | 1 | Glyco | | |
| 4 | DCA | Deoxycholic acid | | 2 | | | |
| | LCA | Lithocholic acid | | 1 | | | |
| | DHCA-S3 | Dihdroxycholanic acid sulfate-3 | | 2 | | Yes | |
| | DHCE-S3 | Dihydroxycholenic acid sulfate-3 | | 2 | | Yes | Yes |
| | MHCA-S2 | Monohydroxycholanic acid sulfate-2 | | 1 | | Yes | |
| 5 | LCA-S | Lithocholic acid sulfate | | 1 | | Yes | |
| | DHCE-S5 | Dihydroxycholenic acid sulfate-5 | | 2 | | Yes | Yes |
| | DHCA-S2 | Dihydroxycholanic acid sulfate-2 | | 2 | | Yes | |
| | DHCA-S5 | Dihydroxycholanic acid sulfate-5 | | 2 | | Yes | |
| 6 | DHCA-S1 | Dihydroxycholanic acid sulfate-1 | | 2 | | Yes | |
| | DHCA-S4 | Dihydroxycholanic acid sulfate-4 | | 2 | | Yes | |
| 7 | DHCE-S1 | Dihydroxycholenic acid sulfate-1 | | 2 | | Yes | Yes |
| | DHCE-S2 | Dihydroxycholenic acid sulfate-2 | | 2 | | Yes | Yes |
| | DHCE-S4 | Dihydroxycholenic acid sulfate-4 | | 2 | | Yes | Yes |
| | MHCA-S1 | Monohydroxycholanic acid sulfate-1 | | 1 | | Yes | |
| | MHCE-S1 | Monohydroxycholanic acid sulfate-1 | | 1 | | Yes | Yes |

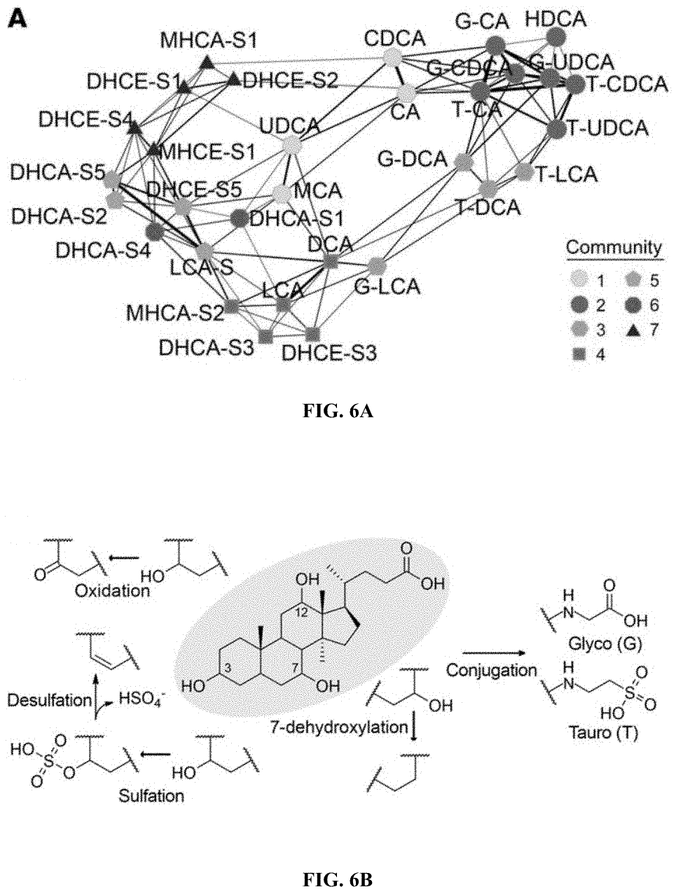

^{a}Bile acids monitored by LC-MS are listed in order of community assignment (FIG. 6).
^{b}Where a reference standard is available to confirm chemical identity, the common name is used. Where lack of a reference standard prevents isomeric assignment, a candidate chemical structure is provided in italics.
^{c}Bile acids are marked “primary” if they have a known host biosynthetic origin.
^{d}Alternatively, structure may include a keto group.

TABLE 4
Instrument settings for detecting bile acids via LC-MS/MS.
Instrument Settings

| | | |
|---|---|---|
| Ion Spray Voltage | −4.5 | kV |
| Heater Temperature | 500° | C. |

| | |
|---|---|
| Nebulizer Gas | 40 |
| | 40 |

| | |
|---|---|
| −12 | V |
| −10 | V |

| Abbreviation | Precursor (m/z) | Product (m/z) | Collision Energy (V) |
|---|---|---|---|
| MCA | 407 | 387 | −45 |
| CA | 407 | 343 | −50 |
| CDCA | 391 | 391 | −50 |
| DCA | 391 | 345 | −50 |
| MHCE-S | 453.8 | 96.9 | −80 |
| DHCE-S | 469.7 | 96.9 | −80 |
| G-CA | 464 | 402 | −50 |
| G-CDCA | 448 | 386 | −50 |
| G-DCA | 448 | 402 | −50 |
| G-LCA | 432 | 388 | −45 |
| G-UDCA | 448 | 386 | −50 |
| HDCA | 391 | 373 | −45 |
| LCA | 375 | 375 | −50 |
| LCA-S | 455.8 | 96.9 | −80 |
| T-CA | 514 | 514 | −50 |
| T-CDCA | 498 | 107 | −80 |
| T-DCA | 498 | 107 | −80 |
| T-LCA | 482 | 107 | −80 |
| T-UDCA | 498 | 107 | −80 |
| UDCA | 391 | 391 | −50 |
| DHCA-S | 471.7 | 96.9 | −80 |

Data preparation. Ion chromatograms were used to align peaks and determine peak areas using Mass Profiler Professional software (Agilent) for GO-MS data and Analyst (AB Sciex) for LC-MS/MS data from the QTrap. Because of the large dynamic range and strong skew of feature intensities, we transformed observed signals at level x to log 10(1+x) values prior to multivariate analyses.

Sparse logistic regression. We use the framework of logistic regression models to classify samples using their measured metabolomic features. Since there are many more metabolomic features than there are samples, we employed multiple measures to avoid overfitting the data. First, we enforced sparsity with an L1 penalty on the number of parameters selected as shown in Equation 1.

$${min}_{w , c} {\sum }_{i} \vert {w}_{i} \vert + C {\sum }_{i}^{n} ( exp ( - {y}_{i} ( {X}_{i}^{T} w + c ) ) + 1 )$$

Equation 1. This analysis is incorporated as part of the python module scikit-learn (Pedregosa F, et al., J Mach Learn Res. 2011-12-2825-2830). The L1 penalty introduces a trade-off between model goodness of fit and the number of incorporated features that is tunable by an additional penalization parameter, C, in Equation 1. Second, we evaluated model performance using repeated 5-fold cross-validation with random subsets to optimize the sparsity penalty, as well as to identify which features were used most frequently and were consistently predictive on the hold-out (testing) sets. Because overfitting on training data is generally expected to reduce performance on a hold-out set, this procedure allowed us to identify the penalization level that maximizes expected performance on the testing set.

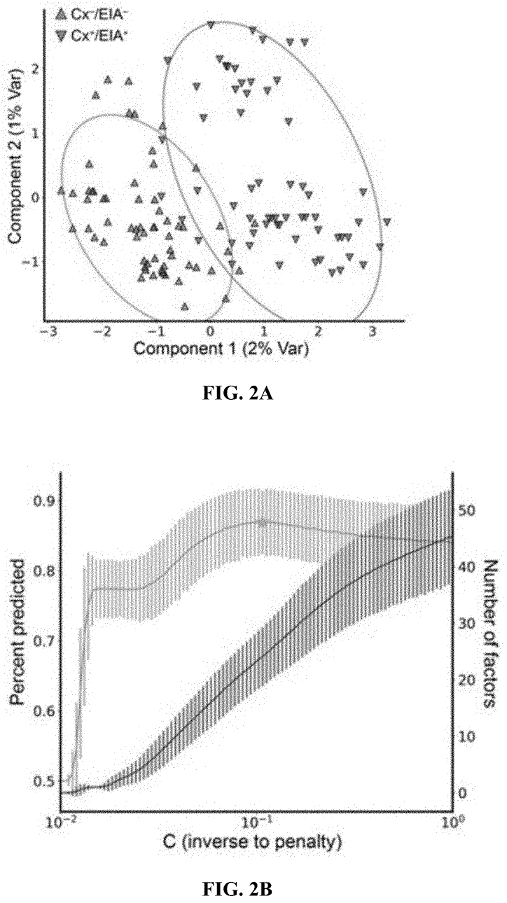

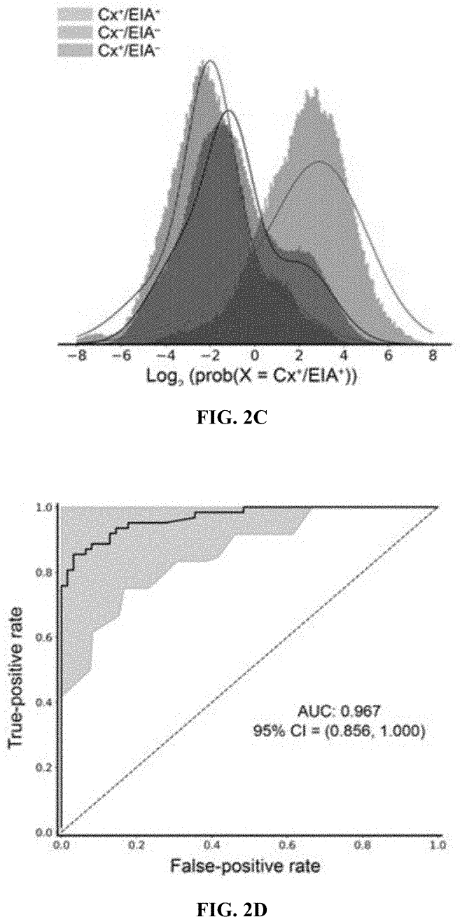

Finally, we obtained the 6-feature logistic regression described in the main text though combination of the results from our repeated 5-fold cross-validated L1-penalized regressions, selecting the 6 metabolomic features most frequently obtained in these sparse logistic regressions. Using only those 6 features, we performed logistic regression (not L1-penalized) on the 124 samples, obtaining the 96.7% AUC in FIG. 2D. We established 95% confidence intervals by further 5-fold cross-validation, keeping the 6 features fixed but varying their coefficient contributions according to each training subset, yielding the 95% CI: 85.6%-100%.

By way of contrast, we compared these results to a logistic regression performed on the full set of features without any sparsity criteria. As expected, since there are an order of magnitude more features than samples, it was possible to select regression coefficients that perfectly separate (AUC=1) the 2 classes in this case. However, this separation is potentially meaningless because of overfitting. Similarly establishing a 95% confidence interval by 5-fold cross-validation, using all metabolomic features, yields the 95% CI: 84.6%-99.5%. While this is still very high—and indeed, is comparable to the CI for our regression using only 6 features—the outlier nature of the artificial perfectly separated result trained using all of the data is a warning of possible overfitting. At the same time, we noted that even with this potential for overfitting using all of the data, it performed no better than our 6-feature regression in terms of CI, while the 6-feature model of course provided much greater ease of interpretation.

We noted that this regression analysis on log-transformed signals does not normalize across samples, nor employ methods to treat the data in a compositional framework, despite the fact that relative abundances of metabolites are the biologically meaningful quantity. Nevertheless, this analysis successfully identifies features whose ratios are informative in predicting classes (see main text).

Sparse partial least squares-discriminatory analysis (sPLS-DA). To further assess the consistency of our data analysis results, we employed sPLS-DA to find a low-rank approximation of the feature data set that aims to maximally preserve the covariance between the dependent variable (EIA status) and the independent variables (the features) (Lê Cao K A, et al., BMC Bioinformatics. 2009; 10:34; Lê Cao K A, et al., Stat Appl Genet Mol Biol. 2008; 7(1):Article 35). This technique identifies a matrix decomposition similar to PCA that best explains the relationship between the variables of interest using the fewest number of features possible. This analysis was conducted using the R package mixOmics (Rohart F, et al., mixOmics: An R package for ‘omics feature selection and multiple data integration. PLoS Comput Biol. 2017; 13(11):e1005752). We conducted both single- and multivariable prediction using sPLS-DA. PLS-DA attempts to find a single decomposition of both the observations and the variable of interest such that the covariance between the projected observations and the projected variables is maximized in the projected space. In this setting with many more features than observations (p>>n), there are typically many low-dimensional combinations of features that can capture variation in the variable of interest; moreover, these combinations will be typically dense in the sense that most features will appear with small but nonzero contributions to prediction. In contrast, the sparse version of PLS-DA, sPLS-DA, simultaneously models the observations while performing feature selection by maximizing the original objective function under conditions to minimize the number of features incorporated.

Network-based analysis of bile acids. The network representation of detected bile acids was defined here using the correlations across all 186 samples as edge weights, keeping the 5 highest positive correlations associated with each bile acid (5 nearest neighbors). Communities were detected from this network using the GenLouvain and CHAMP packages (Weir W H, et al., Algorithms. 2017; 10(3):93; Bastian M, et al. An open source software for exploring and manipulating networks. Accessed Jun. 24, 2019). We selected the obtained 7-community partition for visualization in FIG. 6A, with the network layout produced by the ForceAtlas2 algorithm in Gephi (Weir W H, et al., Algorithms. 2017; 10(3):93; Bastian M, et al. An open source software for exploring and manipulating networks. Accessed Jun. 24, 2019).

Study approval. Approval was obtained from the Washington University Institutional Review Board with a waiver of informed consent to use specimens for this study. Patient and specimen evaluation for this cohort has recently been described (Dubberke E R, et al. Infect Control Hosp Epidemiol. 2018; 39(11):1330-1333).

### Results

### (i) Clinical Cohort.

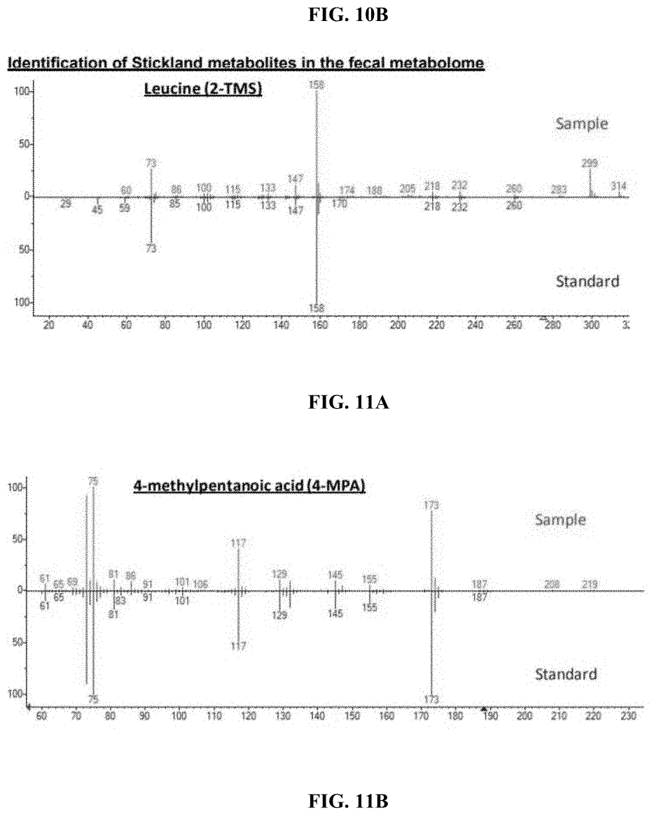

Diarrheal specimens meeting inclusion and exclusion criteria were cultured for C. difficile. All C. difficile isolates recovered in culture were characterized for the presence of toxins tcdA, tcdB, cdtA, and cdtB by multiplex PCR, and underwent PCR ribotyping as previously described (Hink T, et al., Anaerobe. 2013; 19:39-43; Westblade L F, et al., J Clin Microbiol. 2013; 51(2):621-624; Alasmari F et al., Clin Infect Dis. 2014; 59(2):216-222; Dubberke E R, et al., Infect Control Hosp Epidemiol. 2018; 39(11):1330-1333). Of the 8931 available stool specimens (FIG. 10), 2829 were eligible for chart review, through which an additional 2206 were excluded, yielding 622 stool specimens meeting inclusion and exclusion criteria. From these specimens, a 186-person cohort was assembled split into 3 groups of 62 patients matched by age and hospital location. These groups were defined by laboratory results: toxigenic culture-positive and toxin enzyme immunoassay-positive (using the Wample/TechLab Tox A/B II assay during routine clinical testing, Cx⁺/EIA⁺), toxigenic culture-positive and toxin enzyme immunoassay-negative (Cx⁺/EIA⁻), and toxigenic culture-negative and toxin enzyme immunoassay-negative (Cx⁻/EIA⁻) controls. Cohort demographics and clinical characteristics are shown in Table 5.

TABLE 5
Demographic of patient cohorts, including a summary of C. difficile ribotypes

| | | | Asymptomatically | | Not colonized | |
|---|---|---|---|---|---|---|
| | CDI (toxigenic | | colonized (toxigenic | | (toxigenic culture [symbol] , | |
| | culture [symbol] , toxin EIA [symbol] ) | | culture [symbol] , toxin EIA [symbol] ) | | toxin EIA [symbol] ) | |
| Characteristic | n = 62 | | n = 62 | | n = 62 | |
| Ward at stool collection | | | | | | |
| Ward | 50 | (81%) | 50 | (81%) | 50 | (81%) |
| Oncology | 9 | (15%) | 9 | (15%) | 9 | (15%) |
| ICU | 3 | (5%) | 3 | (5%) | 3 | (5%) |
| Age, categorical | | | | | | |
| ≤45 | 10 | (16%) | 17 | (27%) | 8 | (13%) |
| >45-≤65 | 20 | (32%) | 22 | (36%) | 19 | (31%) |
| >65-≤85 | 27 | (44%) | 21 | (34%) | 28 | (45%) |
| >85 | 5 | (8%) | 2 | (3%) | 7 | (11%) |
| Female | 35 | (57%) | 35 | (57%) | 29 | (47%) |
| Race | | | | | | |
| Caucasian | 49 | (79%) | 37 | (60%) | 44 | (71%) |
| African American | 9 | (15%) | 22 | (36%) | 14 | (23%) |
| Other | 0 | (0%) | 1 | (2%) | 1 | (2%) |
| Unknown | 4 | (7%) | 2 | (3%) | 3 | (5%) |

| | | | | | |
|---|---|---|---|---|---|
| Toxin status | | | | | |
| Toxin A | 62 | (100%) | 62 | (100%) | NA |
| Toxin B | 62 | (100%) | 61 | (98%) | NA |
| Binary toxin | 26 | (42%) | 9 | (15%) | NA |
| C. difficile strain [symbol] | | | | | |
| 027 | 22 | (36%) | 8 | (13%) | NA |
| 106/174 | 11 | (18%) | 6 | (10%) | NA |
| WU27 | 6 | (10%) | 1 | (2%) | NA |
| 002 | 4 | (7%) | 3 | (5%) | NA |
| 001 | 4 | (7%) | 6 | (10%) | NA |
| WU8 | 3 | (5%) | 1 | (2%) | NA |
| 014/020 | 2 | (3%) | 9 | (15%) | NA |
| 017 | 2 | (3%) | 2 | (3%) | NA |
| WU2 | 2 | (3%) | 4 | (7%) | NA |
| WU30 | 1 | (2%) | 2 | (3%) | NA |
| WU36 | 0 | (0%) | 4 | (7%) | NA |
| 075 | 0 | (0%) | 4 | (7%) | NA |
| 015/046 | 0 | (0%) | 2 | (3%) | NA |
| 070 | 0 | (0%) | 2 | (3%) | NA |
| Other strain [symbol] | 5 | (8%) | 8 | (13%) | NA |

[symbol]

C. difficile strain is given as ribotype (3 digits), or WU strain type if there was no match to a Cardiff-ECOC collection ribotype strain. [symbol]
Listed as other strain if only a single isolate was recovered. [symbol]
indicates data missing or illegible when filed

### (ii) Fecal Metabolome Characteristics.

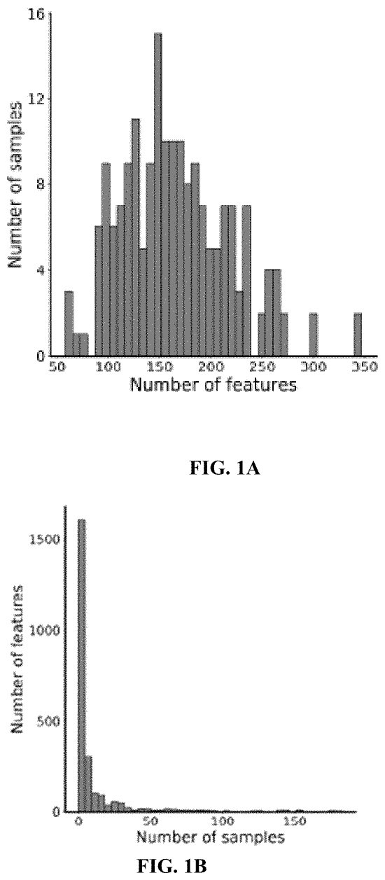

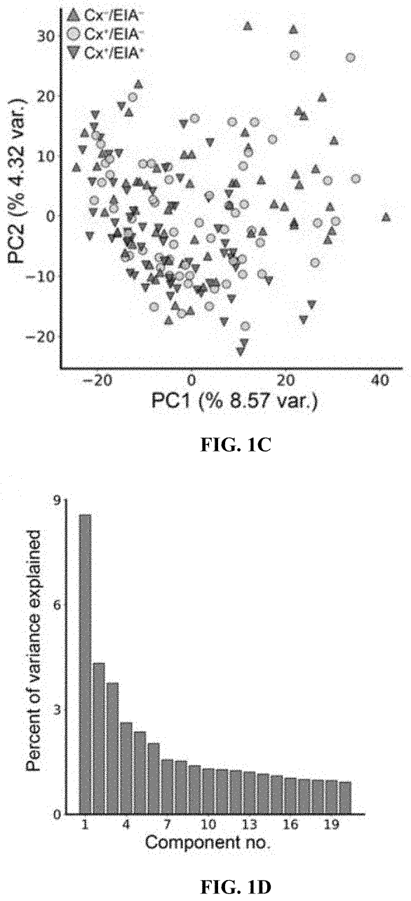

To characterize fecal metabolomic variations in the study cohort, trimethylsilyl-derivatized fecal extracts using GC-MS were detected and quantified. GC-MS is sensitive to low-molecular-weight analytes and does not detect proteins, peptides, complex lipids, or other macromolecules. Ions produced by electron ionization (EI) were detected, which oftentimes provides sufficient structure information to chemically identify metabolites of interest. Fecal metabolites may originate from human cells, microbiome, and/or diet. To compare metabolomes between specimens in the study population, GC-MS profiles were aligned so that each analyte (hereafter called a feature) is defined by its characteristic EI mass spectrum and GC retention time. Within the 186 patient specimens, 2540 distinct features were detected, 77 of which were removed as contaminants because they were present at comparable levels in multiple blank controls, leaving 2463 features for metabolomic analyses. These features were sparsely distributed with a heavy tail (FIG. 1A), with only 593 features appearing in at least 8 (5%) specimens. The number of molecular features per sample was approximately normally distributed (FIG. 1B; mean 164 features, standard deviation 54 features). Principal component analysis (PCA) of log-transformed feature intensities revealed no dominant modes of variation, with the first principal component explaining less than 10% of the overall variance in the data (FIG. 1C and FIG. 1D). Fecal metabolomes defined by GC-MS thus exhibit a high degree of individual variation, with only a small minority of metabolites common to all subjects.

(iii) Metabolomic Differences Between C. difficile-Infected and Uninfected Controls.

To identify CDI-associated fecal metabolites, a supervised multivariate comparison of Cx⁺/EIA⁺ and Cx⁻/EIA⁻ specimens was conducted. Cx⁺/EIA⁺ specimens were used to represent CDI because they harbor viable, toxigenic C. difficile alongside evidence of concurrent toxin production. Given the chemical complexity of fecal metabolomes (the >2000 resolved features greatly exceed the 124 samples), multiple complementary measures were employed to avoid overfitting the data, including repeated cross-validation (see Methods). Sparse partial least squares-discriminatory analysis (sPLS-DA) (FIG. 2A) demonstrates good separation between metabolite profiles from the Cx⁺/EIA⁺ and Cx⁻/EIA⁻ groups, despite this model's use of an explicit penalty to prevent overfitting. To further assess this relationship, a separate logistic regression analysis on the Cx⁺/EIA⁺ and Cx⁻/EIA⁻ groups was conducted with a similar penalization parameter to avoid overfitting. Using repeated 5-fold cross-validation with random subsets to select an appropriate penalization level, it was found that relatively few molecular features yielded a large jump in average accuracy of the regression model (FIG. 2B). We fixed the penalty parameter to the value yielding the maximum percent predicted, indicated by the star in FIG. 2B, and again performed penalized logistic regression fit to the Cx⁺/EIA⁺ and Cx⁻/EIA⁻ groups with repeated randomized 5-fold cross-validation. The observed distributions of log-odds for the test folds (that is, excluding the training sets) for Cx⁺/EIA⁺ and Cx⁻/EIA⁻ again demonstrate good separation (FIG. 2C). For comparison, FIG. 2C also includes the distributions of the log-odds values for the Cx⁺/EIA⁻ cases. The 9 metabolite features most consistently associated with Cx⁺/EIA⁺ specimens (Table 6 and Table 7) include both positive and negative associations. The features consist of 2 SCFAs, 1 amino acid, 1 bile acid, 1 lipid, 3 carbohydrates, and 1 aromatic alcohol. These results implicate biochemically diverse metabolites in human CDI pathogenesis. We then fit a logistic model using only the 6 features that were most frequently selected across the cross-validation runs. This model achieves a ROC AUC (area under the receiver-operator characteristic curve) of 96.7%, with a 95% confidence interval of 85.6%-100% obtained under repeated randomized 5-fold cross-validation (FIG. 2D). These results are consistent with a strong, characteristic signal that distinguishes Cx⁺/EIA⁺ specimens from Cx⁻/EIA⁻ controls.

TABLE 6
Top CDI-associated metabolites during cross-validation of logistic regression model

| Feature | | Median odds | | |
|---|---|---|---|---|
| (mass@RT) | Frequency^{A} | ratio (95% CI) | Metabolite | Class |
| 173.0@4.71 | 100% | 1.54 (1.41-1.69) | 4-methylpentanoic acid, TMS derivative^{B} | SCFA |
| 117.0@4.70 | 99% | 1.16 (1.04-1.30) | 4-methlypentanoic acid, TMS derivative^{B} | SCFA |
| 159.0@7.55 | 99% | 1.12 (1.02-1.25) | 2-hydroxy-4-methlypentanoic acid | SCFA |
| 86.0@6.755 | 98% | 0.91 (0.84-0.98) | Isoleucine | Amino acid |
| 215.0@24.80 | 96% | 0.87 (0.78-0.98) | Cholenoic acid | Bile acid |
| 73.0@13.79 | 95% | 0.92 (0.83-0.99) | Ribitol | Carbohydrate |
| 67.0@20.17 | 94% | 1.11 (1.01-1.24) | Eicosatrienoic acid | Lipid |
| 179.0@12.06 | 94% | 1.15 (1.02-1.27) | Tyrosol, 2TMS derivative | Aromatic alcohol |
| 204.0@19.45 | 91% | 0.87 (0.77-0.98) | Glyceryl glycoside | Carbohydrate |
| 217.0@14.72 | 89% | 0.93 (0.84-0.99) | Fructose | Carbohydrate |

^{A}Proportion of cross-validation models including the metabolite.
^{B}Two ions from same metabolite were independently resolved.

TABLE 7
CDI-associated metabolites from cross-validation of logistic regression model.

| | | Median Odds | |
|---|---|---|---|
| Mass@RT | Frequency ^{A} | Ratio (95% CI) | Compound |
| 173.0@4.71 | 100% | 1.54 (1.40-1.68) | 4-methylpentanoic acid, TMS derivative ^{B} |
| 117.0@4.70 | 99% | 1.19 (1.04-1.30) | 4-methylpentanoic acid, TMS derivative ^{B} |
| 159.0@7.55 | 99% | 1.13 (1.02-1.25) | 2-hydroxy-4-methylpentanioc acid |
| 86.0@6.75 | 98% | 0.91 (0.84-0.98) | isoleucine |
| 215.0@24.80 | 96% | 0.87 (0.77-0.98) | cholenoic acid (bile acid BA1) |
| 73.0@13.79 | 95% | 0.92 (0.83-0.99) | ribitol |
| 67.0@20.17 | 94% | 1.14 (1.02-1.27) | eicosatrienoic acid |
| 179.0@12.06 | 94% | 1.11 (1.01-1.23) | tyrosol, 2 TMS derivative |
| 204.0@19.45 | 91% | 0.87 (0.76-0.99) | glyceryl glycoside |
| 217.0@14.72 | 89% | 0.92 (0.84-0.99) | fructose |
| 73.0@11.41 | 83% | 1.09 (1.00-1.13) | L-S-oxoproline, 2 TMS derivative |
| 73.0@11.32 | 81% | 1.09 (1.00-1.13) | trans-4-hydroxycyclohexanecarboxylic acid |
| 255.0@27.45 | 80% | 0.99 (0.88-1.00) | hydroxycholenoic acid (bile acid BA2) |
| 73.0@14.37 | 69% | 0.96 (0.88-1.00) | no match |
| 73.0@11.12 | 68% | 0.96 (0.89-1.00) | no match |
| 205.0@8.12 | 63% | 0.94 (0.86-1.00) | glycerol, 3 TMS derivative |
| 194.0@10.01 | 63% | 0.96 (0.50-1.00) | 194.0@ 10.021012 |
| 79.0@19.99 | 59% | 1.04 (1.00-1.14) | arachidonic acid, TMS derivative |
| 142.0@12.16 | 57% | 0.96 (0.88-1.00) | no match |
| 73.0@15.99 | 56% | 0.97 (0.90-1.00) | D-sorbitol, 6 TMS derivative |
| 217.0@12.74 | 47% | 0.97 (0.50-1.00) | D-[−]-ribofuranose, tetrakis(trimethylsilyl) ether (isomer 2) |
| 75.0@6.77 | 43% | 1.03 (1.00-1.13) | indole-3-acetic acid |
| 202.0@16.29 | 43% | 1.03 (1.00-1.11) | no match |
| 73.0@16.17 | 33% | 0.97 (0.50-1.00) | no match |
| 145.0@6.51 | 30% | 1.03 (1.00-1.12) | 2-hydroxy-3-methylbutyric acid, 2 TMS derivative |
| 255.0@25.45 | 25% | 0.97 (0.50-1.00) | deoxycholic acid (bile acid BA3) |
| 158.0@8.35 | 24% | 1.03 (1.00-1.13) | allo-isoleucine, 2 TMS derivative |
| 84.0@11.56 | 24% | 0.98 (0.92-1.00) | no match |
| 73.0@8.90 | 23% | 0.98 (0.91-1.00) | glyceric acid, 3 TMS derivative |
| 217.0@15.71 | 22% | 0.98 (0.92-1.00) | meglumine, 5 TMS derivative |
| 204.0@15.65 | 22% | 0.98 (0.92-1.00) | no match |
| 147.0@9.95 | 21% | 0.98 (0.91-1.00) | D-[−]-erythrofuranose, tris(trimethylsilyl) ether (isomer 2) |
| 204.0@15.13 | 20% | 0.98 (0.92-1.00) | no match |
| 73.0@12.53 | 18% | 1.03 (1.00-1.12) | alpha-arabinopyranose, 4 TMS derivative |
| 179.0@14.22 | 18% | 0.98 (0.92-1.00) | 3-(4-hydroxyphenyl)propanoic acid (phloretic acid) ^{C} |
| 73.0@12.86 | 18% | 0.98 (0.92-1.00) | no match |
| 230.0@11.47 | 16% | 0.98 (0.90-1.00) | no match |
| 73.0@12.39 | 16% | 0.98 (0.91-1.00) | no match |
| 73.0@11.98 | 16% | 0.98 (0.93-1.00) | no match |
| 174.0@15.81 | 14% | 1.02 (1.00-1.08) | tyramine |
| 151.0@4.93 | 13% | 0.98 (0.90-1.00) | phenol, TMS derivative |
| 86.0@6.44 | 12% | 0.98 (0.92-1.00) | L-leucine, TMS derivative |
| 73.0@14.90 | 11% | 0.99 (0.92-1.00) | no match |

^{A} Proportion of cross-validation models including the metabolite
^{B} Two ions from same metabolite were independently resolved
^{C} This is also referred to as desamino tyrosine

### (iv) Stickland Amino Acid Fermentation in CDI.

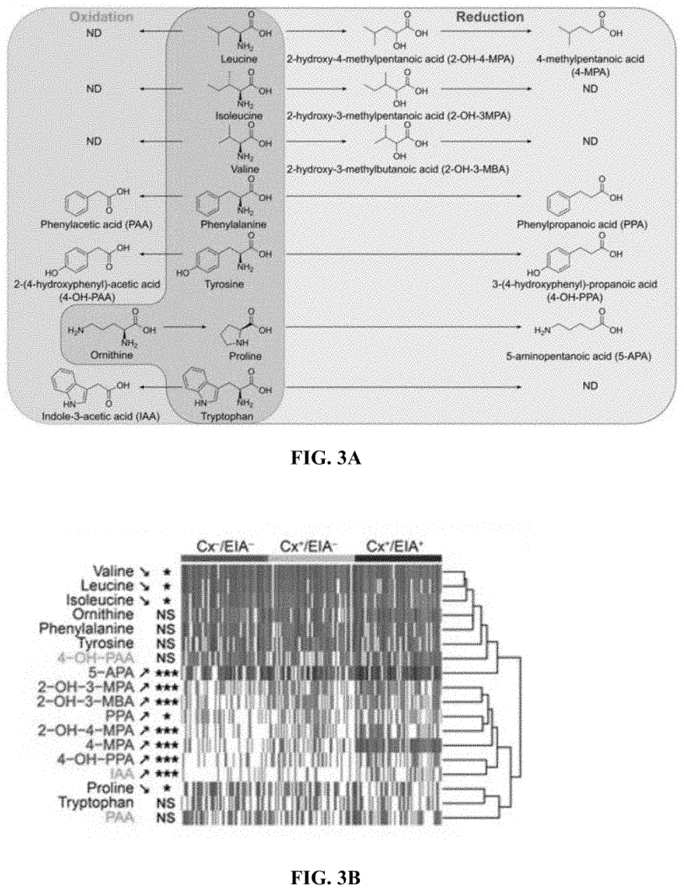

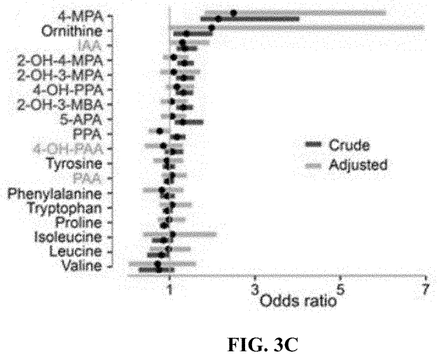

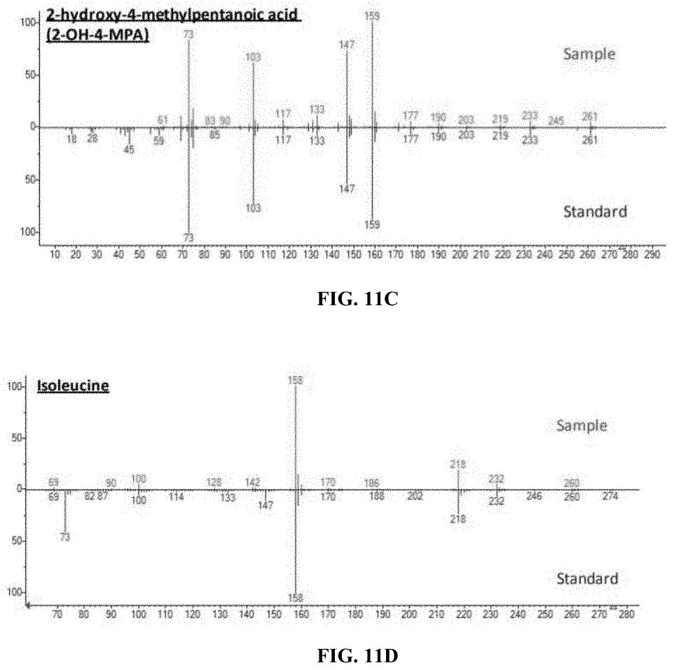

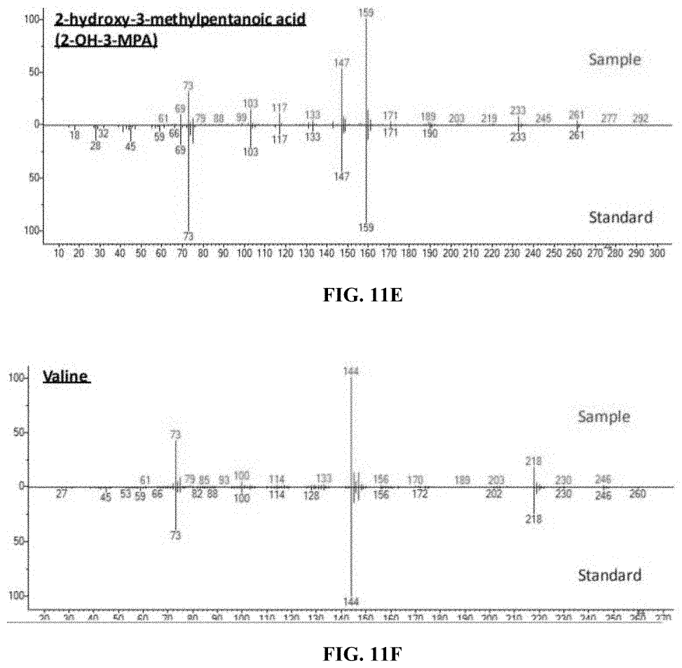

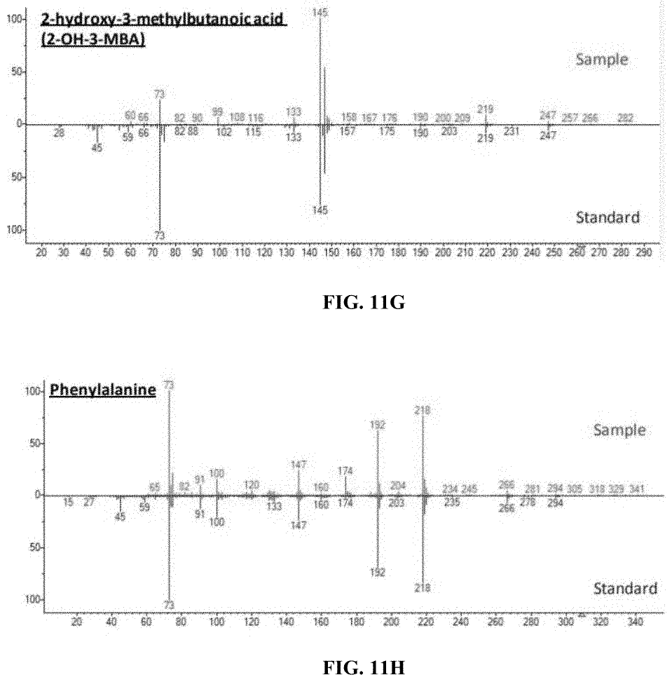

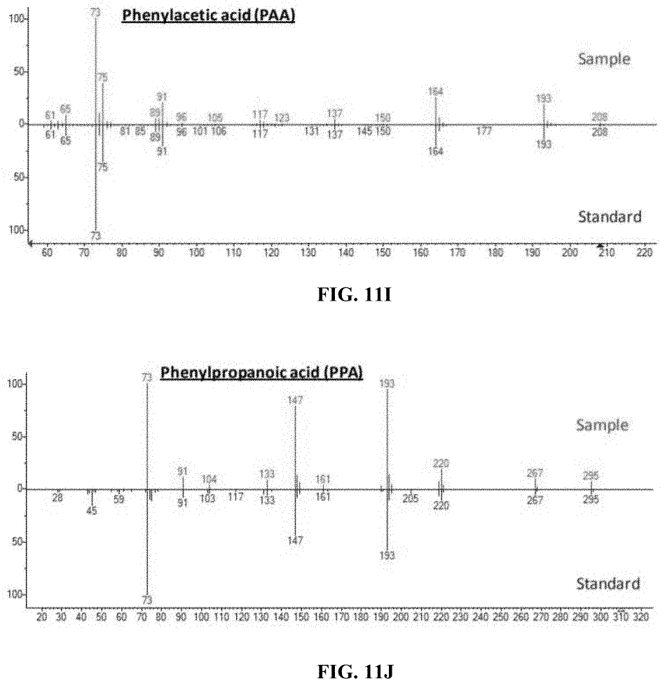

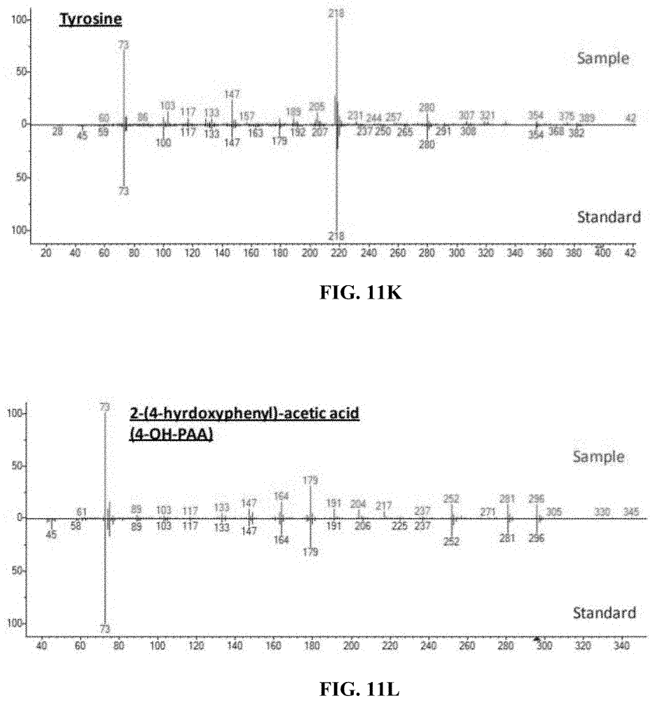

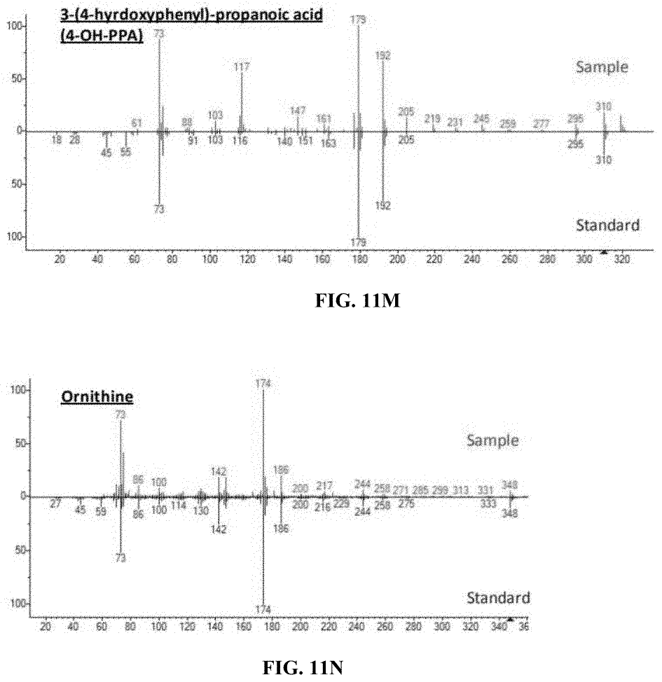

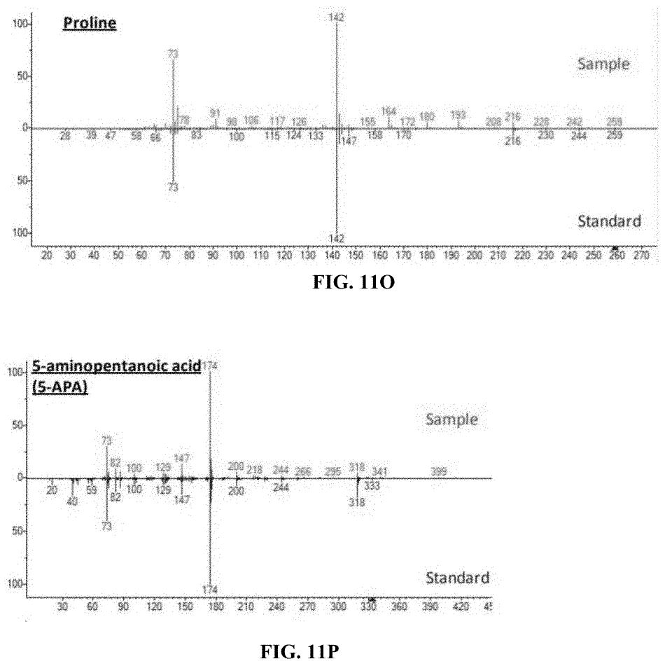

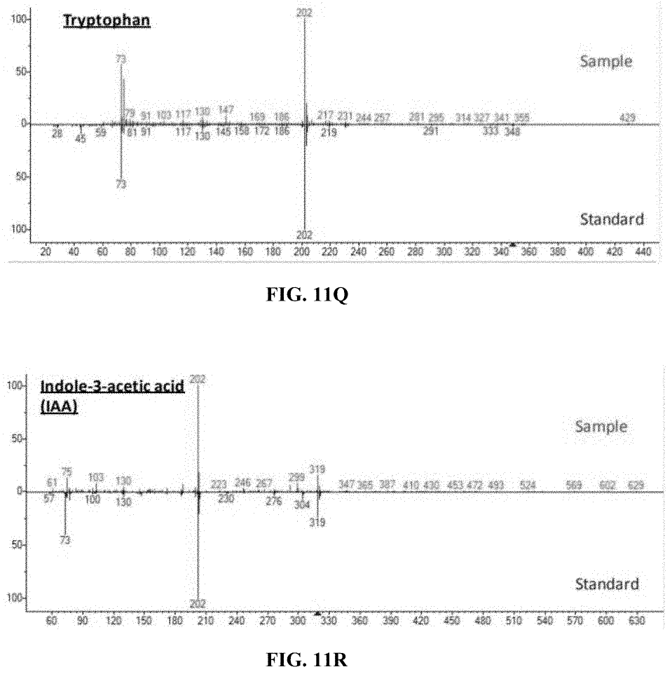

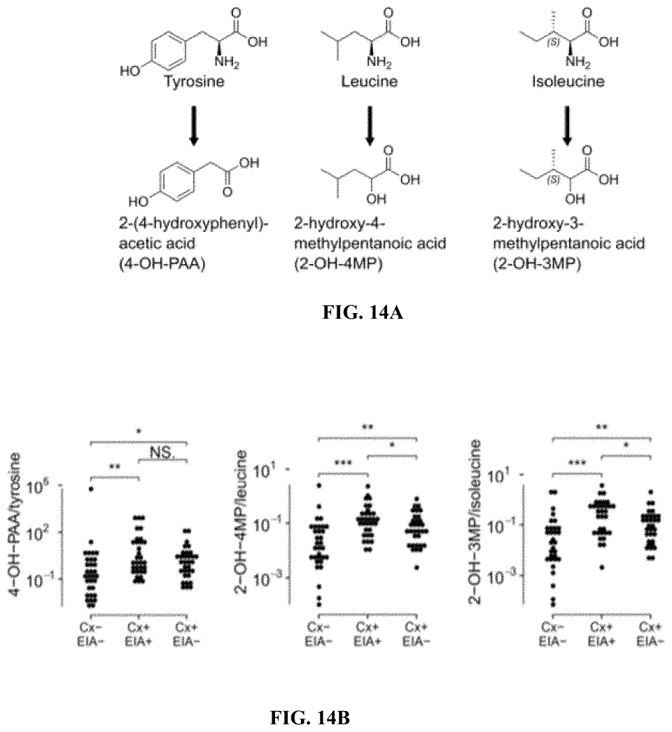

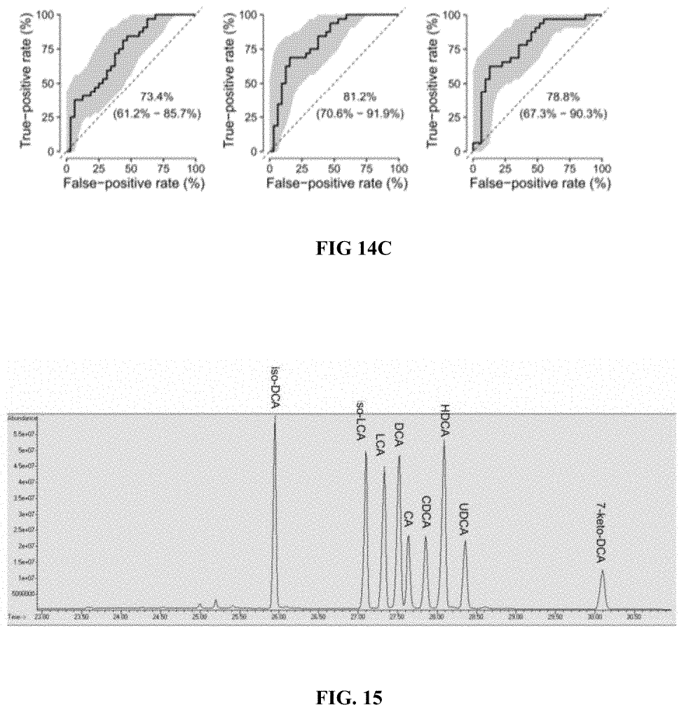

Among the most highly CDI-associated metabolites (Table 6) is the SOFA 4-methylpentanoic acid (4-MPA/4-methylvaleric acid/isocaproic acid). Unlike the SCFAs formate, acetate, and butyrate, which are produced during microbial carbohydrate fermentation, 4-MPA is produced from leucine through the Stickland reactions, amino acid fermentation pathways associated with C. difficile and other anaerobic bacteria (Stickland L H, Biochem J. 1934; 28(5):1746-1759; Stickland L H, Biochem J. 1935; 29(4):889-898; Nisman B, Bacteriol Rev. 1954; 18(1):16-42; Neumann-Schaal M, et al., Front Microbiol. 2019; 10:219; Kim J, et al., Appl Environ Microbiol. 2006; 72(9):6062-6069; Britz M L, et al., Can J Microbiol. 1982; 28(3):291-300; Elsden S R, et al., Arch Microbiol. 1978; 117(2):165-172; Elsden S R, et al., Arch Microbiol. 1976; 107(3):283-288; Bouillaut L, Self W T, Sonenshein A L. Proline-dependent regulation of Clostridium difficile Stickland metabolism. J Bacteriol. 2013; 195(4):844-854; Dyer J K, et al., J Bacteriol. 1968; 96(5):1617-1622). Ten established Stickland products were detected in the study cohort, representing both oxidative and reductive fermentation of 8 different amino acid precursors (FIG. 3A and FIG. 11). These products exhibit varying degrees of association with CDI, with 8 of 10 products (80%) detected more frequently in CDI specimens than controls (FIG. 3B and FIG. 14). Many Stickland products were present in Cx⁻/EIA⁻ specimens, consistent with production by bacteria other than toxigenic C. difficile. Bootstrapped logistic regression (fit on 2000 bootstrap samples, stratified on Cx/EIA status) of Stickland metabolites consistently assigns the highest odds ratios for CDI to 4-MPA, the end product of leucine reduction (FIG. 3C). Although other canonical Stickland products like 5-aminopentanoic acid (5-aminovaleric acid) are frequently present in CDI, they offer negligible discriminatory power beyond that of 4-MPA in the adjusted analysis.

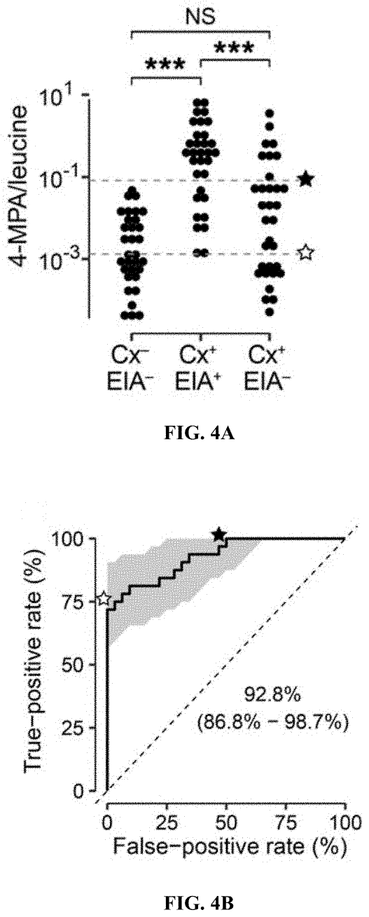

To more precisely quantify the relationship between 4-MPA production and CDI, a targeted GC-MS assay was devised to quantify Stickland fermentation activity through product/precursor ratios. In addition to increasing assay sensitivity and precision, this targeted biomarker ratio is intrinsically insensitive to the variations in fecal dilution that characterize diarrheal specimens. In an arbitrary subset of matched specimens, the 4-MPA/leucine ratio varied significantly between groups (P=1.3×10⁻⁸, Kruskal-Wallis test). This variation distinguishes Cx⁺/EIA⁺ specimens from Cx⁻/EIA⁻ specimens with an ROC AUC of 92.8% (95% CI: 86.8%-98.7%; FIG. 4A and FIG. 4B) that rivals the 6-feature regression model described above and in the Methods (FIG. 2D; AUC=96.7%; 95% CI: 85.6%-100%).

Together, these results are consistent with a pathophysiologic role for Stickland fermentation in CDI. While the presence of these metabolites in Cx⁻/EIA⁻ specimens suggests that intestinal Stickland metabolism in patients is not generally unique to CDI, the selective increase in 4-MPA in CDI specimens raises the possibility that leucine reduction is a selectively emphasized pathway in C. difficile during clinical infections.

(v) The Isomeric Amino Acid Allo-Isoleucine is Associated with CDI.

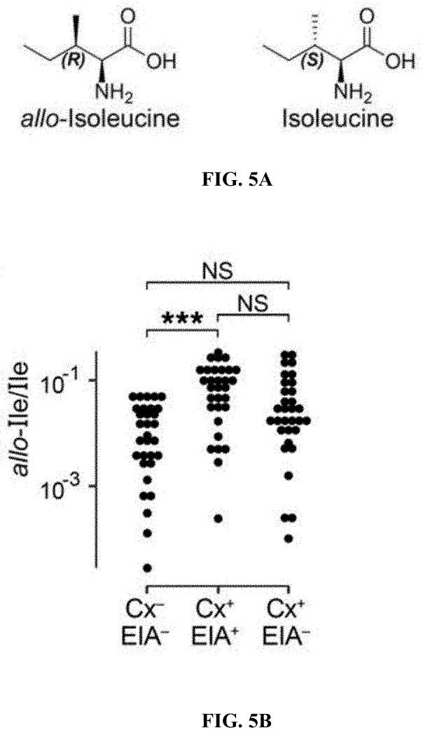

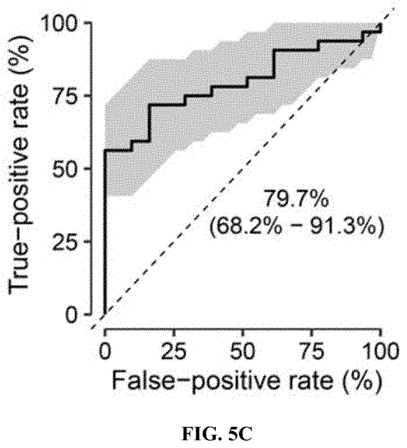

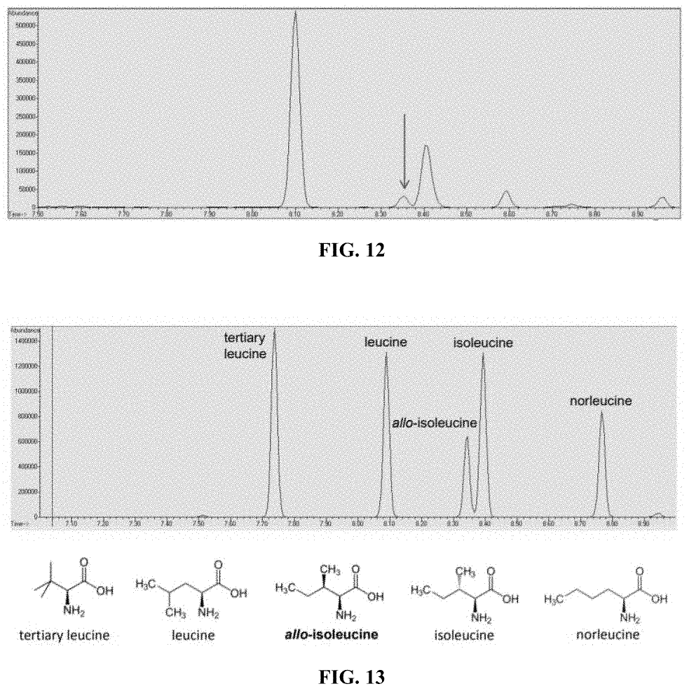

Among the metabolites that are positively associated with CDI is allo-isoleucine, an isoleucine diastereomer in which the beta carbon stereocenter is inverted from an S to an R configuration (FIG. 5A). This noncanonical, nonproteinogenic amino acid has been identified as a biomarker of branched chain ketoaciduria (maple syrup urine disease, an inborn error of metabolism) but has not previously been associated with C. difficile or CDI. Its origins in feces are unclear, although a previously reported bacterial metabolic pathway producing it from L-isoleucine raises the possibility that it derives from the intestinal microbiome (Li Q, et al., J Am Chem Soc. 2016; 138(1):408-415). To more carefully assess the relationship between allo-isoleucine and CDI, a targeted GC-MS assay was devised to quantify allo-isoleucine as a ratio to isoleucine, its putative precursor. The allo-isoleucine-to-isoleucine ratio varied significantly between groups (P=6.5×10-5, Kruskal-Wallis test; FIG. 5B and FIG. 12 and FIG. 13). ROC analysis (FIG. 5C) (AUC=79.7%; 95% CI: 68.2%-91.3%) suggested favorable diagnostic potential for distinguishing Cx⁺/EIA⁺ specimens from Cx⁻/EIA⁻ specimens. These observations identify allo-isoleucine as a new and biochemically distinctive CDI correlate of unclear origin.

(vi) Bile Acid Metabolic Pathways Active in Patients without CDI

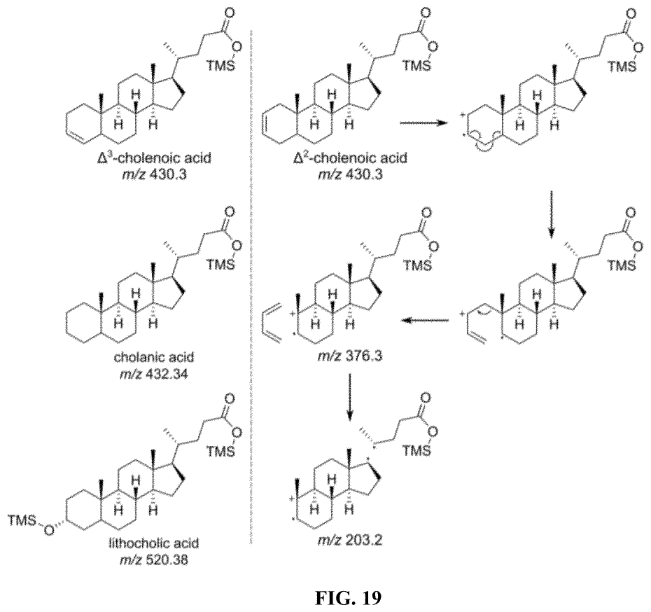

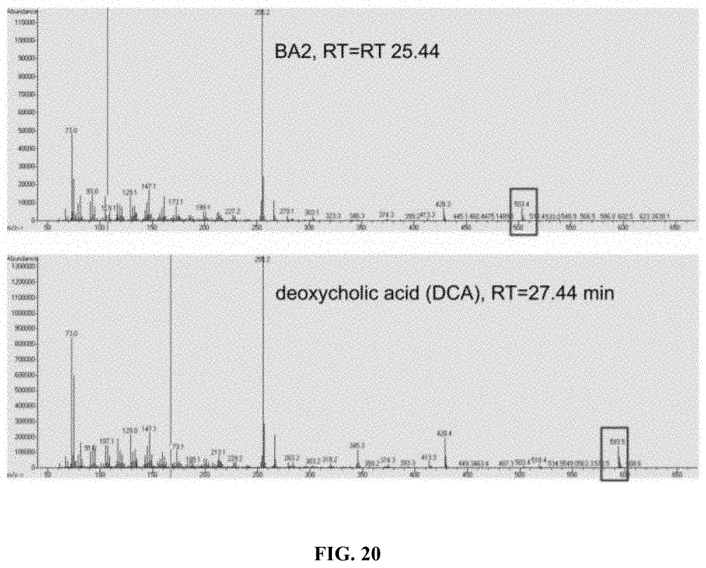

Three negatively loaded bile acid features are among the most frequently detected Cx⁺/EIA⁺ correlates in our cross-validated analysis (Table 6 and Table 7). This corresponds to previous scholarship, which has associated bile acid dehydroxylation by the intestinal microbiota with CDI susceptibility (Khoruts A, et al. Nat Rev Gastroenterol Hepatol. 2016; 13(9):508-516; Palmieri L J, et al., Front Microbiol. 2018; 9:2849; Brown J R, et al., BMC Gastroenterol. 2018; 18(1):131, Seekatz A M, et al., Anaerobe. 2018; 53:64-73). Canonical bile acid processing by the microbiome involves successive dehydroxylation of cholic acid (CA; a tri-hydroxylated primary bile acid) to deoxycholic (DCA, a di-hydroxylated secondary bile acid) and chenodeoxycholic acid (CDCA; a di-hydroxylated primary bile acid) to lithocholic acid (LCA, a mono-hydroxylated secondary bile acid). Unexpectedly, the 2 most highly CDI-associated bile acids in our cohort were identified as cholenoic acid and monohydroxycholenoic acid (CE and MHCE, respectively, FIGS. 15-20), noncanonical unsaturated, dehydroxylated bile acids. As with DCA and LCA, these bile acids were more abundant in the non-CDI group, consistent with an alternative bile acid dehydroxylation pathway based on dehydration reactions (net loss of H₂O to yield a double bond).

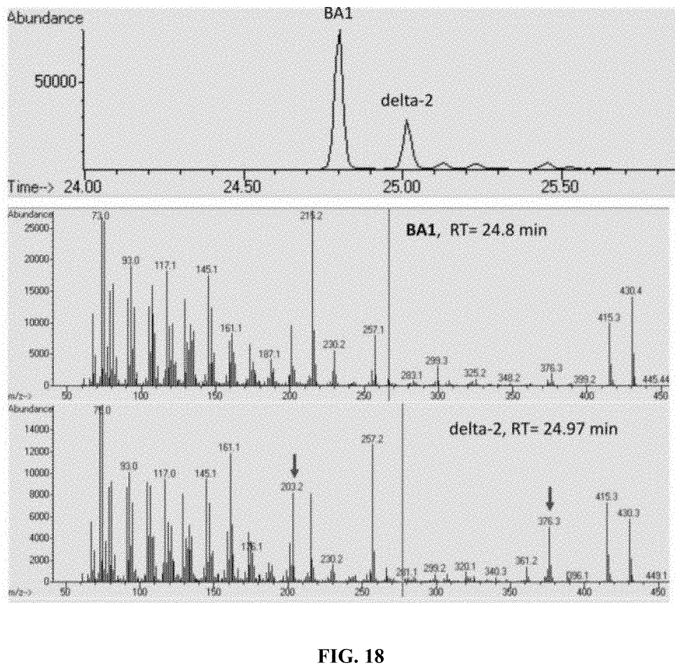

Unsaturated, nonhydroxylated bile acids are seldom considered in the bile acid literature. Their absence from our metabolite database compelled us to identify them through manual interpretation of spectra and comparison to chemically related reference compounds (FIGS. 15-20). CE, a nonhydroxylated, unsaturated bile acid, was previously identified by Robben et al. as a lithocholic acid sulfate (LCA-S) desulfation product generated by an intestinal isolate of the Bacteroidaceae family (Robben J, et al. Appl Environ Microbiol. 1989; 55(11):2954-2959). Robben et al. noted 2 isomeric CE products of these bacteria that differ in double bond location. We similarly observed 2 closely eluting CE products, consistent with a similar product distribution in our patient cohort (FIG. 18). Human tissues are known to generate sulfated bile acids, including LCA-S, which may provide substrates for fecal CE production through enzymatic desulfation (Hofmann A F, et al., Drug Metab Rev. 2004; 36(3-4):703-722). These observations are consistent with diminished microbial bile acid desulfation activity in patients with CDI.

(vii) Identification of a CDI-Associated Human Bile Acid Network.

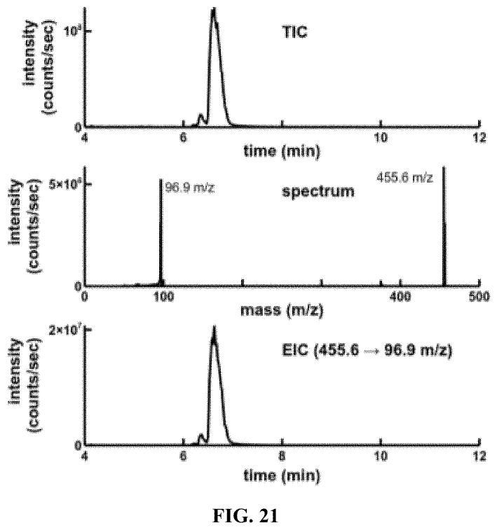

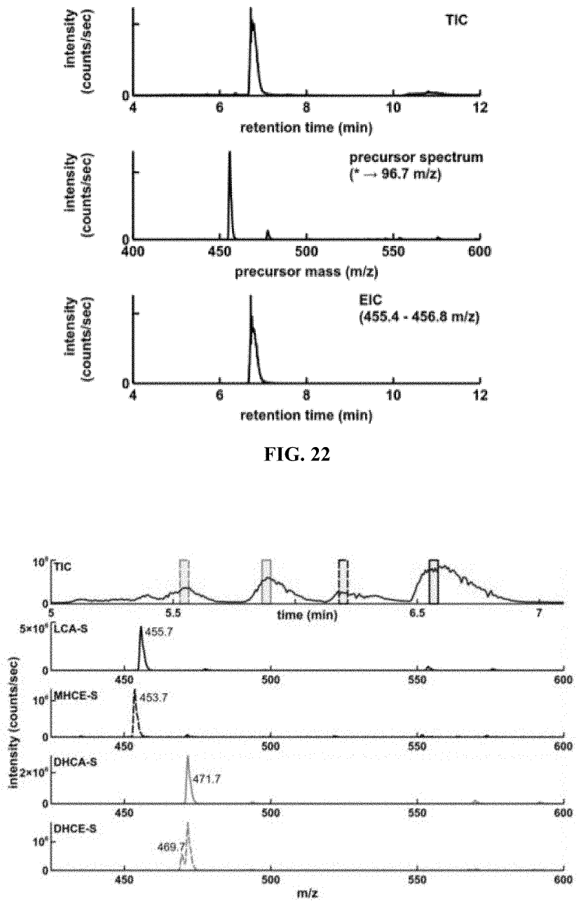

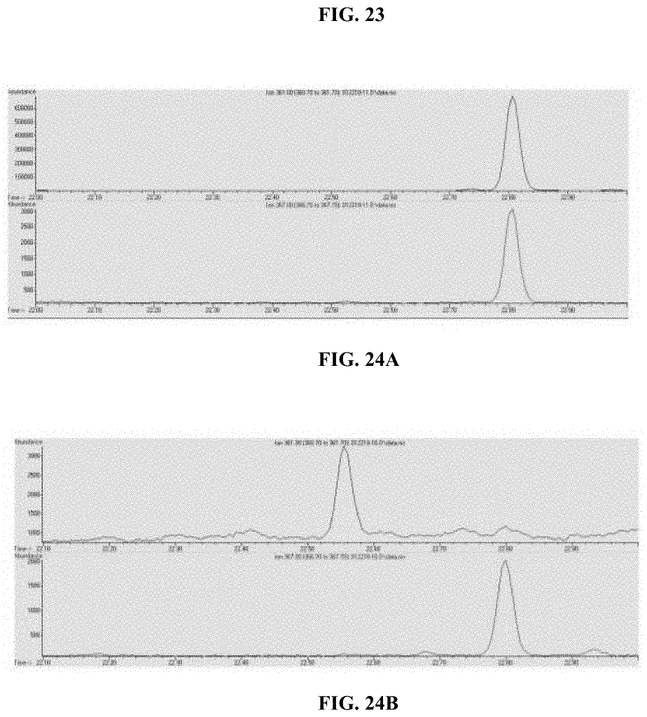

Based on the presence of CE and MHCE in patient specimens, it was hypothesized that sulfated bile acids (the precursors of unsaturated bile acids) (Hofmann A F, et al., Drug Metab Rev. 2004; 36(3-4):703-722) are also present. It was further hypothesized that the desulfation mechanism of unsaturated bile acid production is generalizable such that an extended series of bile acid sulfates and unsaturated bile acids are present in the human fecal metabolome (FIG. 6B). Using the calculated molecular weights, MS/MS fragmentation patterns, and chromatographic elution ranges for these hypothesized bile acids, a liquid chromatography-tandem mass spectrometry (LC-MS/MS) assay was constructed (FIGS. 21-23 and Table 4) because sulfated bile acids are undetectable by GC-MS. This assay resulted in tentative detection of 14 sulfated bile acids, 6 of which were dehydrogenated (possessing either an alkene or ketone; Table 3). Many of these bile acids are distinguishable only by retention time, consistent with isomers that differ in the position(s) of double bonds, hydroxyl groups, and/or sulfate.

Although fecal bile acids largely originate from 2 primary bile acids (CA and CDCA), subsequent host conjugation, divergent microbiome cometabolism, and enterohepatic circulation create a complex, nonlinear bile acid physiology. To characterize bile acid interrelationships, community detection (Weir W H, et al. CHAMP package: Convex Hull of Admissible Modularity Partitions, Accessed Jun. 24, 2019) was performed on the weighted network of positive correlations among the 14 noncanonical bile acids described above and 17 canonical conjugated and nonconjugated primary and secondary bile acids. Seven bile acid communities emerged from this unbiased network community detection analysis, many of which could be rationalized by shared chemical features (Table 3 and FIG. 6A). Where unavailability of authentic internal standards prevents identification of hydroxylation sites (e.g., the 3, 7, and 12 carbon positions) or epimers, bile acids are designated with general names. Communities 1 to 3 are composed exclusively of canonical primary and secondary bile acids. Community 1 consists of classic primary bile acids while community 2 consists of their glycine or taurine conjugates. Community 3 consists of conjugated secondary (dehydroxylated) bile acids. Community 4 includes secondary bile acids, secondary bile acid sulfates, and 1 candidate di-hydroxylated cholenic acid sulfate. Communities 5 and 6 consist entirely of sulfated bile acids, with a single sulfated cholenic acid candidate. The 5 bile acids in community 7 are all sulfated, with 4 cholenic acid sulfate candidates. The 5 candidate dehydroxylated cholenic acid sulfates may plausibly include sulfated keto bile acids, secondary bile acids of identical mass. In a force-directed layout depicting this network (FIG. 6A), the primary bile acids (CA, CDCA) are located centrally, consistent with their recognized roles as precursors to conjugated and secondary bile acids. Clockwise progression moves from bile acid communities defined by host glycine and taurine conjugation, to classical microbial dehydroxylation, to sulfation, to desaturation or ketone formation (FIG. 6B). The community organization emerging from this analysis reflects the distinctive metabolic transformations identified in the present study and in previous work.

(viii) Bile Acid Metabolomic Associations with CDI.

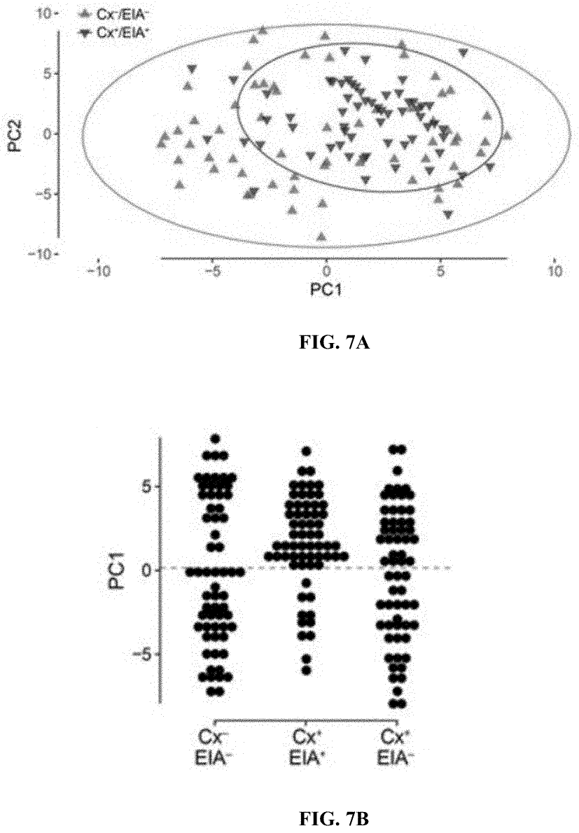

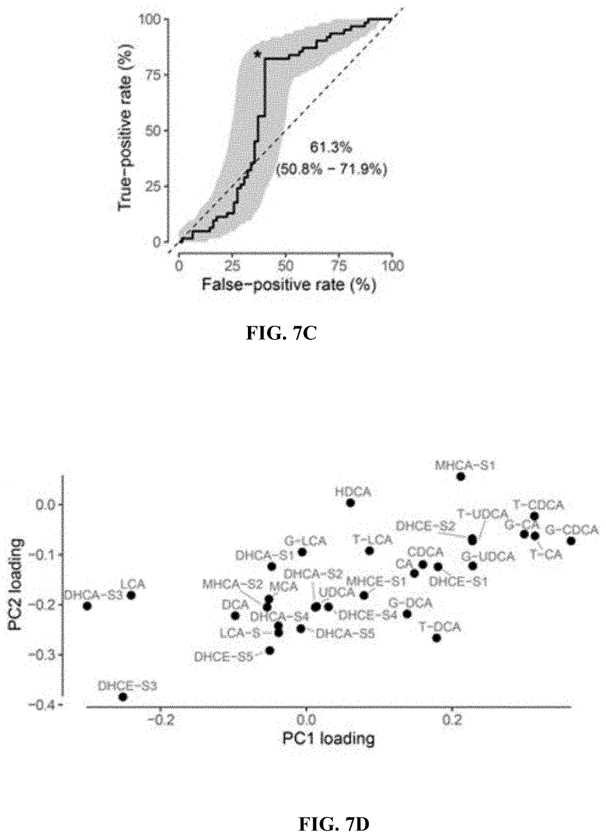

Disruption of microbiome-mediated bile acid metabolism has long been regarded to increase CDI risk. In our inpatient cohort, it was hypothesized that the Cx⁻/EIA⁻ group includes a subset of patients with disrupted, CDI-susceptible microbiomes. To test this hypothesis, we used PCA to graphically summarize bile acid metabolomic variation in culture-negative specimens (FIG. 7A and FIG. 7B). Next, we projected Cx⁺/EIA⁺ bile acid profiles onto these principal components. Consistent with the hypothesis, Cx⁺/EIA⁺ specimens preferentially occupied a restricted portion of the Cx⁻/EIA⁻ patient bile acid profile distribution. Specifically, Cx⁺/EIA⁺ specimens preferentially exhibit elevated values along the first PCA-derived principal component (PC1). High PC1 scores correspond to higher primary (cholic and chenodeoxycholic) and low secondary (deoxycholic and lithocholic) bile acids (FIG. 7D), similar to previous studies (Brown J R, et al., BMC Gastroenterol. 2018; 18(1):131; Seekatz A M, et al., Anaerobe. 2018; 53:64-73). Low PC1 scores correspond to higher levels of sulfated and dehydroxylated cholenic and cholanic acids (DHCA-S3, DHCE-S3, LCA from community 4). ROC analysis using PC1 as the discriminator revealed an AUC of 61.3% (FIG. 7C). These results are consistent with a negative association between CDI and bile acid sulfation, dehydroxylation, and unsaturation. While we cannot conclude a causative role from these correlative data, these metabolic processes may indicate the presence of a CDI-resistant intestinal microbiome.

(ix) Fecal Carbohydrate Associations with CDI.

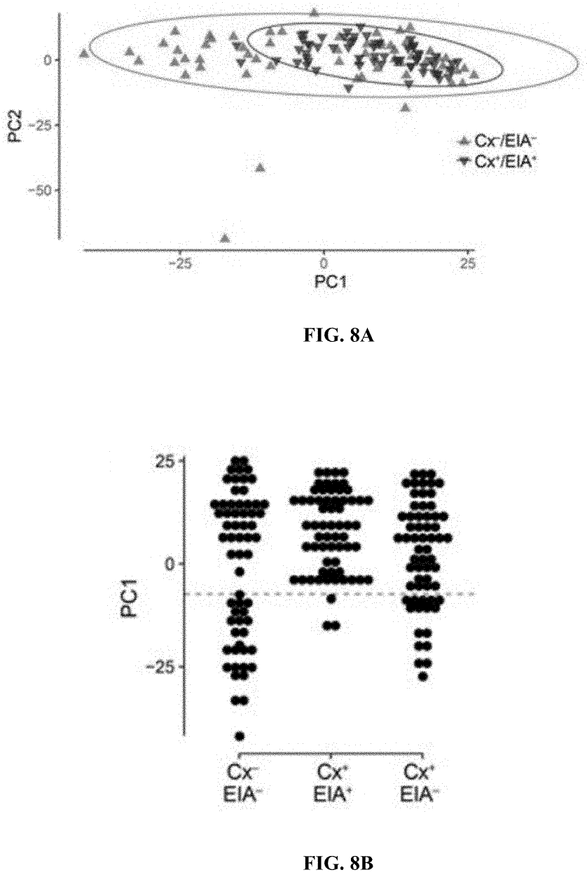

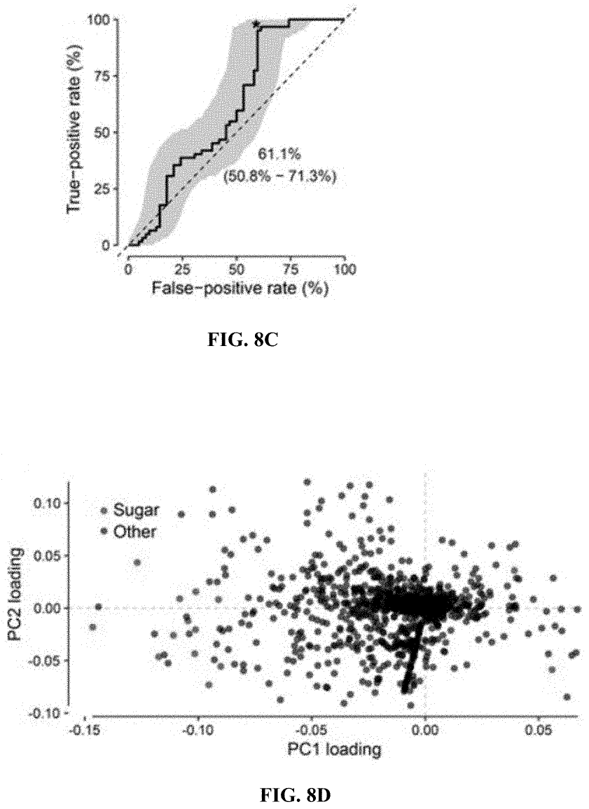

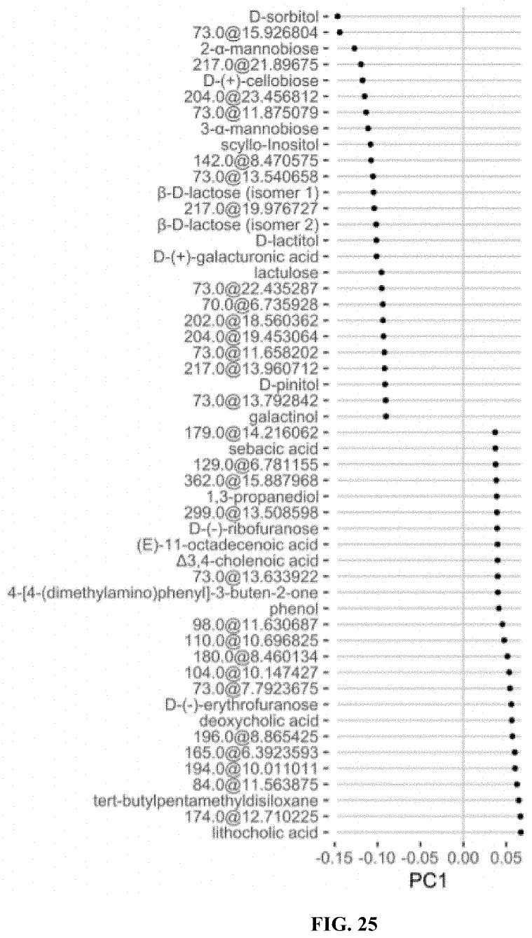

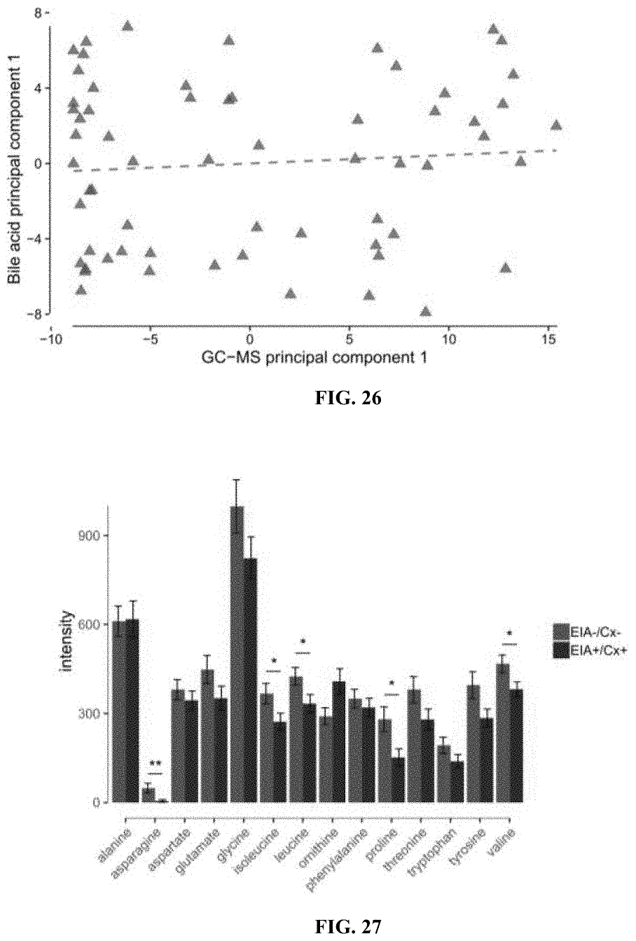

It was next hypothesized that the Cx⁻/EIA⁻ group includes patients with CDI-susceptible intestinal metabolites other than bile acids. To test this hypothesis, we used PCA to graphically summarize total GC-MS detectable metabolomic variation in culture-negative specimens. Next, we projected CDI patient metabolomes onto these principal components. Consistent with the hypothesis, CDI patient fecal metabolomes occupy a restricted portion of the uncolonized patient distribution, characterized by a high PC1 score (FIG. 8A and FIG. 8B). ROC analyses of PC1 scores yielded a modest AUC of 61.1% when distinguishing Cx⁺/EIA⁺ from Cx⁻/EIA⁻ specimens (FIG. 8C). These metabolites are not clearly related to bile acid composition, since the total metabolome PC1 exhibits a low degree of association with the bile acid PC1 determined above (r²<0.007; FIG. 26). Instead, high PC1 scores are primarily characterized by diminished monosaccharides, disaccharides, and sugar alcohols with uncertain relationships to CDI (FIG. 8D and FIG. 25). While these metabolite classes can be reasonably identified by GC-MS, identifying specific isomers is often unreliable (e.g., sorbitol and mannitol are both C₆H₁₄O₆ and differ only in the orientation of 1 hydroxyl group and yield comparable spectra). The monosaccharide fructose, a favored C. difficile carbon substrate (Edwards A N et al., Methods Mol Biol. 2016; 1476:117-128), emerged as a negative CDI correlate in the logistic regression analysis above (Table 6), raising the possibility that some carbohydrates may be consumed by metabolically active C. difficile. Trehalose, a disaccharide recently reported to be a favored substrate of epidemic C. difficile ribotypes 027 and 078, was not identified in our differential analysis (Collins J, et al. Nature. 2018; 553(7688):291-294). To more carefully assess the relationship between trehalose and CDI, we quantified fecal trehalose using a targeted GC-MS analysis based on stable isotope dilution with a ¹³C6-labeled internal standard (FIG. 24). It was detectable in 61% (115/189) of specimens but did not distinguish Cx⁺/EIA⁺ from Cx⁻/EIA⁺ specimens (35/63 vs. 41/63, P=0.36, 2-tailed Fisher's exact test). In 027-positive specimens, trehalose also did not distinguish toxin-positive from toxin-negative specimens (6/8 vs. 12/23, P=0.41, 2-tailed Fisher's exact test). A subset of fecal carbohydrates thus has some potential to distinguish CDI and possibly CDI-susceptible patients, though the basis for this remains unclear.

### (x) A Metabolomic Model of CDI.

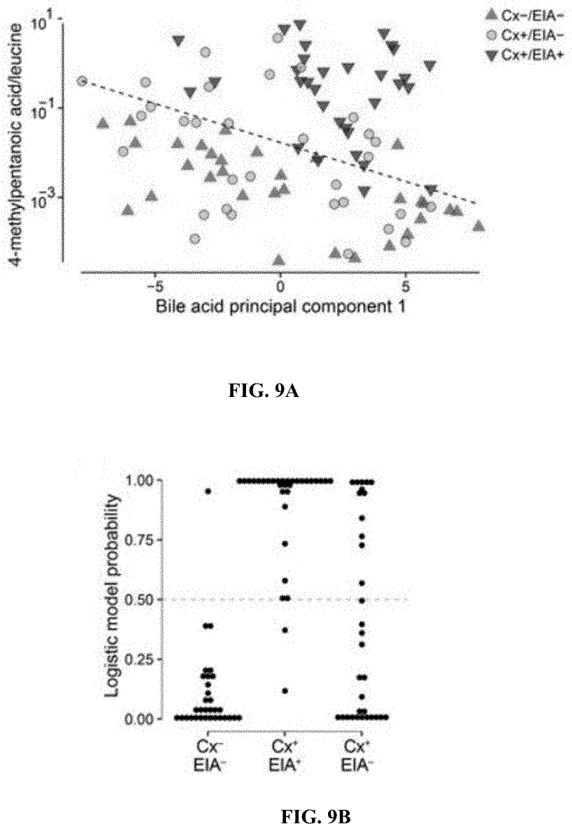

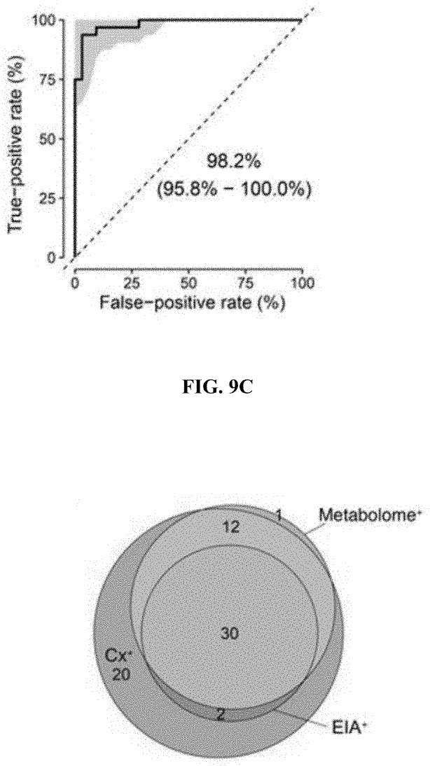

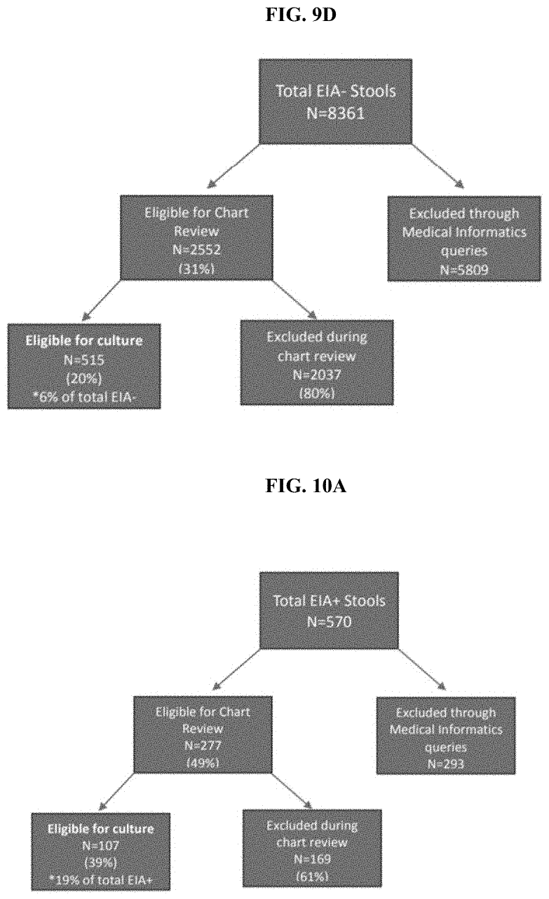

To determine whether fecal Stickland metabolites and bile acids can be used to construct a metabolomic definition of CDI, we conducted logistic regression using the 4-MPA/leucine ratio (log₁₀-transformed) and the bile acid PC1 (Table 8 and FIG. 9A). Each parameter alone exhibited significant (P<0.05) independent associations with Cx⁺/EIA⁺ status when compared with Cx⁻/EIA⁻ specimens. When the logistic model criterion is applied (corresponding to >50% probability), Cx⁺/EIA⁺ specimens clustered in the high 4-MPA/leucine and high bile acid PC1 quadrant (FIG. 9A and FIG. 9B). ROC analysis of this model yields an AUC of 98.2%, out-performing the original 6-feature model described above (FIG. 9C). Each parameter contributed independently-adding a term for interaction between 4-MPA/leucine ratio and bile acid PC1 did not significantly improve the logistic model (P=0.53, analysis of deviance). These results are consistent with distinctive host and microbial metabolic processes in human CDI.

TABLE 8
Logistic Regression Model of CDI Metabolome

| | Crude odds | | |
|---|---|---|---|
| Parameter | ratio (95% CI) | Adjusted | P value^{A} |
| Log₁₀ (4-MPA index) | 2.09 (2.01-6.06) | 4.06 (2.15-7.53) | <0.0001 |
| Bile acid PC1 | 0.15 (0.01-0.30) | 0.71 (0.31-1.42) | 0.033 |

^{A}Analysis of deviance comparison between single-parameter logistic regression model and the null logistic model.

(xi) Metabolomic Differences in Colonized Patients with and without Detectable Fecal Toxin.

To determine whether Cx⁺/EIA⁻ specimens possess distinctive metabolomes, we compared 4-MPA/leucine and bile acid composition profiles from Cx⁺/EIA⁻ specimens to those of Cx⁺/EIA⁺ or Cx⁻/EIA⁻ specimens. In the logistic regression model, only 38% (20/32) resembled Cx⁺/EIA⁺ specimens, with the remainder exhibiting low 4-MPA/leucine ratios in specimens with or without susceptible bile acid profiles (FIG. 9A and FIG. 9B). These observations are consistent with low C. difficile metabolic activity and a protective bile acid profile in many patients with undetectable fecal toxin. Using the logistic regression parameter compared with toxigenic culture or toxin EIA results alone defines a positive test group that is smaller than (but almost entirely encompassed by) toxigenic culture-positive specimens but greater than the number of toxin EIA-positive specimens (FIG. 9D). If the metabolic criterion is highly accurate, it may restrict false-positive results from toxigenic C. difficile detection alone and also restrict false-negative results from the toxin EIA test. Further study is necessary to determine whether this possibility can be realized.

Discussion

In this example, the fecal metabolomic profiles from 186 hospitalized patients were compared to investigate relationships between fecal metabolites, the presence of toxigenic C. difficile, and the presence of detectable C. difficile toxins. Untargeted metabolomic profiling in the context of uncontrolled patient dietary and microbiome contributions yielded extremely diverse fecal metabolomes. Nevertheless, numerous CDI-associated metabolites were resolved. Among the 2463 features detected in this cohort, 43 had some ability to resolve CDI from uncolonized controls. Many of these discriminatory molecules are associated with Stickland and bile acid metabolism, processes previously implicated in CDI pathogenesis (Khoruts A et al., Nat Rev Gastroenterol Hepatol. 2016; 13(9):508-516; Palmieri L J, et al., Front Microbiol. 2018; 9:2849; Theriot C M, et al., mSphere. 2016; 1(1):e00045-15; Thanissery R, et al., Anaerobe. 2017; 45:86-100; Neumann-Schaal M, et al., Front Microbiol. 2019; 10:219; Kim J, et al., Appl Environ Microbiol. 2006; 72(9):6062-6069; Bouillaut L, et al., J Bacteriol. 2013; 195(4):844-854; Brown J R, et al., BMC Gastroenterol. 2018; 18(1):131; Seekatz A M, et al., Anaerobe. 2018; 53:64-73; Wilson K H, et al., J Clin Microbiol. 1983; 18(4):1017-1019; Battaglioli E J, et al., Sci Transl Med. 2018; 10(464):eaam7019). The specific molecular signatures best able to resolve CDI from controls exhibit only partial overlap with those identified in prior metabolomic studies using mouse models, which may reflect species differences, the presence of a variable host microbiome background, and the specific mass spectrometric approach. Toxin-negative, toxigenic C. difficile-positive (Cx⁺/EIA⁻) specimen metabolomes span a metabolomic continuum ranging from control-like to CDI-like. Among Cx⁺ specimens, fecal metabolites have the potential to distinguish infected from colonized patients.

Identification of 4-MPA as the most prominent CDI correlate is consistent with its production by C. difficile from leucine during Stickland metabolism. Other Stickland products were also detected and observed to be elevated in patients with CDI, although their abundance among the control specimens (Cx⁻/EIA⁻) diminished some of their associations (low positive predictive value), especially that of 5-aminopentanoic acid. This contrasts with previously reported murine studies in which multiple Stickland metabolites are highly CDI-associated. The discrepancy between patient and mouse studies likely arises from Stickland-metabolizing organisms in Cx⁻/EIA⁻ patient microbiomes, which may be limited or absent in the antibiotic-treated mice used in experimental CDI models. 4-MPA has not been uniformly identified as a CDI correlate in metabolomic studies of murine CDI. This may reflect host-associated substrate selection of leucine for Stickland metabolism by toxin-producing C. difficile but may also reflect lack of detection due to the apparent insensitivity of typical untargeted LC-MS approaches to 4-MPA (unpublished observations). Indeed, GC-MS remains a favored modality for SCFA analyses by many investigators. Nevertheless, the implication of Stickland fermentation in CDI is generally consistent with previous human and animal model studies.

The association between Stickland fermentation and CDI is consistent with the hypothesis that fecal amino acid availability enhances CDI susceptibility. Our data do not rule out an important role for carbohydrate metabolism, the C. difficile fermentation products of which (pyruvate, formate, acetate, butyrate) are less distinctive than Stickland metabolites (Neumann-Schaal M, et al., Front Microbiol. 2019; 10:219). Although we observed no association between CDI and fecal trehalose, a glucose disaccharide generated during bacterial stress responses, utilized as a food additive, and proposed as a dietary risk factor for CDI caused by hypervirulent strains (ribotype 027), the other fecal carbohydrates detected in this study may plausibly serve as metabolic substrates (Collins J, et al. Nature. 2018; 553(7688):291-294). A recent study by Battaglioli et al. observed a broad spectrum increase in fecal amino acid concentrations in gnotobiotic mice colonized with dysbiotic human gut microbiota. This increase corresponded to high fecal C. difficile colonization after experimental challenge (Battaglioli E J, et al., Sci Transl Med. 2018; 10(464):eaam7019). In the present example, amino acids tend to be diminished in CDI specimens compared with controls (FIG. 3B and FIG. 3C, and FIG. 27). This apparent contradiction may be reconciled by interpreting the decrease in amino acids during CDI as evidence of consumption by metabolically active C. difficile, which yields the aforementioned Stickland products. The importance of amino acid substrate selection by C. difficile during clinical CDI remains unclear. The present data are consistent with a preference for branched chain amino acids (leucine, isoleucine, and valine) relative to other intestinal microbes, though it is possible that other Stickland substrates, such as proline, tyrosine, phenylalanine, and ornithine, could substitute for branched chain amino acid deficiencies. If so, gut microbiota that deplete a broad range of fecal amino acids may help hosts resist CDI.

In addition to implicating C. difficile metabolic pathways in CDI patients, the present example also identifies a series of CDI-associated bile acids. Previous mouse model studies have identified associations between diminished fecal secondary bile acids and increased C. difficile fecal colonization, which agrees with the general findings of the current study (Khoruts A et al., Nat Rev Gastroenterol Hepatol. 2016; 13(9):508-516; Palmieri U, et al., Front Microbiol. 2018; 9:2849; Theriot C M, et al., mSphere. 2016; 1(1):e00045-15; Thanissery R, et al., Anaerobe. 2017; 45:86-100). Differences in specific bile acids between this study and murine studies likely reflect both species differences (murine bile acids exhibit substantial 6-hydroxylation compared with humans) and different analytical approaches. Human cells synthesize and chemically conjugate bile acids, whereas intestinal microbes have been shown to modify them through dehydroxylation at the 7-carbon position to yield deoxycholic and lithocholic acids (from cholic and chenodeoxycholic acids, respectively). Here, unbiased detection of 2 cholenic acids (cholenic and hydroxycholenic acids, FIG. 15-20) by GC-MS profiling as the most highly CDI-associated bile acids raises the possibility that beneficial microbes can also dehydroxylate bile acids at the 3-carbon position, leaving behind unsaturated, nonhydroxylated bile acids. Detection of monohydroxycholenic acid sulfate (MHCE-S1) provides additional evidence of this pathway. Five additional bile acid sulfate candidates may represent either cholenic acids or keto-bile acids, both of which would exhibit ions 2 mass units below their canonical counterparts. It remains unclear whether cholenic acids are solely CDI-negative patient biomarkers or whether their formation protects patients from CDI (Wilson K H, et al., J Clin Microbiol. 1983; 18(4):1017-1019). Production of these bile acids might confer CDI protection through consumption of progermination bile acids or by direct inhibition of C. difficile spore germination. Additional experimental work is necessary to evaluate these possibilities and could help identify desirable microbiome constituents for future therapeutic strategies.

The biochemical signatures resolved in this study suggest a metabolomic model of human CDI. In addition to identifying therapeutic strategies, such a model may also identify new or refined diagnostic approaches to appropriately identify patients who would benefit from treatment. Current diagnostic approaches are based on nucleic acid-based detection of toxigenic C. difficile and immunoassay-based detection of fecal toxin, each of which raise valid concerns over their associated false-positive and false-negative rates (McDonald L C, et al., Clin Infect Dis. 2018; 66(7):987-994; 10, 36). The metabolomic profiles identified in the current work are biochemically distinct from existing tests and, in a multistep diagnostic approach with existing tests, could improve diagnostic accuracy. Detection of Stickland metabolites would be consistent with the presence of antibiotic-responsive, vegetatively growing C. difficile. Moreover, individualized metabolomic information on whether an unfavorable bile acid profile is present could guide microbiome-directed interventions such as fecal transplant or probiotic administration. The signatures identified here may aid larger patient studies aimed at assessing the value of this approach.

In summary, this metabolomic study suggests specific host, pathogen, and microbiome factors associated with CDI pathogenesis. Strengths of this study include use of a valid clinical study population with relevant control specimens and comparison to clinically accessible test results, use of an unbiased screening approach, use of multiple mass spectrometric methods, and use of strategies to avoid the overfitting issues inherent in many comparative metabolomic approaches. The uniquely high chromatographic resolution and informative electron ionization spectra of GC-MS analysis was likely essential to our detection of 4-MPA, allo-isoleucine, and cholenoic acid, analytes that are poorly detected or resolved under typical LC-MS conditions. Moreover, the ability to identify metabolites using spectrally rich EI fragmentation spectra in GC-MS allowed us to place our analytic findings within a broader biological context.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method of identifying a subject having a Clostridioides difficile infection (CDI), the method comprising:
(i) measuring levels of one or more analytes selected from the group consisting of a short chain fatty acid, an amino acid, a bile acid, a carbohydrates, an aromatic alcohol and a lipid in a biological sample obtained from the subject;
(ii) determining an analyte signature based on the expression levels of the analytes in step (i); and
(iii) identifying CDI occurrence of the subject based on the analyte signature determined in step (ii).

2. The method of claim 1, wherein the biological sample is a fecal sample.

3. The method of claim 1, wherein the analytes are one or more of 4-methylpentanoic acid (4-MPA), 2-hydroxy-4-methylpentanoci acid, isoleucine, leucine, allo-isoleucine, cholenoic acid, ribitol, eicosatrienoic acid, tyrosol, glyceryl glycoside, 4-MPA/leucine ratio and fructose.

4. The method of any one of claims 1-3, wherein the wherein the analyte levels are measured by mass spectrometry.

5. The method of anyone of the proceeding claims, wherein the subject is identified as having or at risk for CDI and the method further comprises subjecting the subject to a treatment for CDI.

6. The method of anyone of the proceeding claims, wherein the subject has undergone a prior treatment for a bacterial infection.

7. The method of any of the proceeding claims, wherein increased analyte levels of 4-methylpentanoic acid (4-MPA), 2-hydroxy-4-methylpentanoci acid, or allo-isoleucine in step (i) relative to a reference value indicates the subject has CDI.

8. The method of any of the proceeding claims, wherein decreased analyte levels of isoleucine, leucine, cholenoic acid, ribitol, eicosatrienoic acid, tyrosol, glyceryl glycoside, or fructose relative to a reference value of a subject having transient synovitis indicates the subject has CDI.

9. The method of any of the proceeding claims, wherein increased analyte levels of 4-methylpentanoic acid (4-MPA), 2-hydroxy-4-methylpentanoci acid, or allo-isoleucine and decreased analyte levels of isoleucine, leucine, cholenoic acid, ribitol, eicosatrienoic acid, tyrosol, glyceryl glycoside, or fructose in step (i) relative to a reference value indicates the subject has CDI.

10. The method of any of the proceeding claims, wherein similar analyte levels of 4-methylpentanoic acid (4-MPA), 2-hydroxy-4-methylpentanoci acid, or allo-isoleucine in step (i) relative to a reference value indicates the subject does not have CDI or is colonized with CDI.

11. The method of any of the proceeding claims, wherein similar analyte levels of isoleucine, leucine, cholenoic acid, ribitol, eicosatrienoic acid, tyrosol, glyceryl glycoside, or fructose relative to a reference value of a subject does not have CDI or is colonized with CDI.

12. A method of treating a subject having a Clostridioides difficile infection (CDI), the method comprising:
(i) measuring levels of one or more analytes selected from the group consisting of a short chain fatty acid, an amino acid, a bile acid, a carbohydrates, an aromatic alcohol and a lipid in a biological sample obtained from the subject;
(ii) determining an analyte signature based on the expression levels of the analytes in step (i);
(iii) assessing CDI occurrence or severity of the subject based on the analyte signature determined in step (ii); and
(iv) treating the subject with an anti-CDI therapeutic when the analyte signature in step (i) relative to a reference value indicates the subject has CDI.

13. The method of claim 12, wherein the biological sample is a fecal sample.

14. The method of claim 12, wherein the analytes are one or more of 4-methylpentanoic acid (4-MPA), 2-hydroxy-4-methylpentanoci acid, isoleucine, leucine, allo-isoleucine, cholenoic acid, ribitol, eicosatrienoic acid, tyrosol, glyceryl glycoside, 4-MPA/leucine ratio and fructose.

15. The method of anyone of claims 12-14, wherein the subject has undergone a prior treatment for a bacterial infection.

16. The method of any one of claims 12-15, wherein the wherein the analyte levels are measured by mass spectrometry.

17. The method of anyone of claims 12-16, wherein increased analyte levels of 4-methylpentanoic acid (4-MPA), 2-hydroxy-4-methylpentanoci acid, or allo-isoleucine in step (i) relative to a reference value indicates the subject has CDI.

18. The method of anyone of claims 12-17, wherein decreased analyte levels of isoleucine, leucine, cholenoic acid, ribitol, eicosatrienoic acid, tyrosol, glyceryl glycoside, or fructose relative to a reference value of a subject having transient synovitis indicates the subject has CDI.

19. The method of anyone of claims 12-18, wherein increased analyte levels of 4-methylpentanoic acid (4-MPA), 2-hydroxy-4-methylpentanoci acid, or allo-isoleucine and decreased analyte levels of isoleucine, leucine, cholenoic acid, ribitol, eicosatrienoic acid, tyrosol, glyceryl glycoside, or fructose in step (i) relative to a reference value indicates the subject has CDI.
